(19) United States
(12) Patent Application Publication
(10) Pub. No.: US 2025/0002659 A1
(43) Pub. Date: Jan. 2, 2025

(54) METHOD OF PREPARING HYALURONIC ACID-ALGINATE (HA-ALG) HYDROGEL AND HA-ALG HYDROGEL

(71) Applicant: Supansa YODMUANG, Bangkok (TH)

(72) Inventors: Supansa YODMUANG, Bangkok (TH); Thanh Nguyên TRÚC, Bangkok (TH)

(73) Assignee: Supansa YODMUANG, Bangkok (TH)

(21) Appl. No.: 18/688,159

(22) PCT Filed: Sep. 2, 2022
(86) PCT No.: PCT/TH2022/050005

§ 371 Date: Feb. 29, 2024

Related U.S. Application Data
(60) Provisional application No. 63/240,262, filed on Sep. 2, 2021.

(51) Int. Cl. C08J 3/075; A61L 27/26; A61L 27/38; A61L 27/52

(52) U.S. Cl. CPC C08J 3/075; A61L 27/26; A61L 27/38; A61L 27/52; C08J 2305/08; C08J 2405/04

(57) ABSTRACT

Disclosed is a method of preparing a Hyaluronic acid-Alginate (HA-Alg) hydrogel, the method comprising: dissolving a required amount of amine-hyaluronic acid (HA-NH₂) in a first buffer solution to form an HA-NH₂ solution of a first predefined concentration; dissolving a required amount of aldehyde-alginate (Alg-CHO) in a second buffer solution to form an Alg-CHO solution of a second predefined concentration; and mixing the HA-NH₂ solution and the Alg-CHO solution in a predefined ratio for preparing the HA-Alg hydrogel.

### TECHNICAL FIELD

The present disclosure relates to methods of preparing Hyaluronic acid-Alginate (HA-Alg) hydrogels. The present disclosure also relates to Hyaluronic acid-Alginate (HA-Alg) hydrogels.

### BACKGROUND

Hydrogels have become very popular due to their properties and have been widely used in many applications ranging from industrial applications to biological applications. The properties of the hydrogels which makes it suitable for various application include high-water content, softness, flexibility, biocompatibility, and the like. Some of the applications of the hydrogels include, manufacturing of contact lenses, hygiene products, tissue engineering scaffolds, drug delivery systems, wound dressings, and the like. Hydrogels may be produced by natural and synthetic hydrophilic polymers by physically or chemically crosslinking them. Hydrogels possess a degree of flexibility very similar to natural tissue owing to their significant water content, therefore have been widely used to encapsulate cells to be used in regenerative tissue engineering.

However, there are several problems associate with conventional hydrogels, in terms of gelation time, cytotoxicity, mechanical strength, shear-thinning properties, and the like. Firstly, some of the conventional hydrogels take longer gelation time to be transformed into a gel. For example, the gelation time may be up to a few hours, which drastically decreases usability of the conventional hydrogels. Secondly, some of the conventional hydrogels prove to be toxic for the cells owing to presence of additive(s), therefore are not suitable for encapsulating the cells, limiting their usability for the regenerative tissue engineering. Thirdly, some of the conventional hydrogels do not exhibit shear thinning properties, which makes them less suitable for printing.

Therefore, in light of the foregoing discussion, there exists a need to overcome the aforementioned drawbacks associated with the conventional hydrogels.

### SUMMARY

The present disclosure seeks to provide a method of preparing a Hyaluronic acid-Alginate (HA-Alg) hydrogel. The present disclosure also seeks to provide a Hyaluronic acid-Alginate (HA-Alg) hydrogel. An aim of the present disclosure is to provide a solution that overcomes at least partially the problems encountered in prior art.

In one aspect, an embodiment of the present disclosure provides a method of preparing a Hyaluronic acid-Alginate (HA-Alg) hydrogel, the method comprising:
dissolving a required amount of amine-hyaluronic acid (HA-NH₂) in a first buffer solution to form an HA-NH₂ solution of a first predefined concentration;
dissolving a required amount of aldehyde-alginate (Alg-CHO) in a second buffer solution to form an Alg-CHO solution of a second predefined concentration; and
mixing the HA-NH₂ solution and the Alg-CHO solution in a predefined ratio for preparing the HA-Alg hydrogel.

In another aspect, an embodiment of the present disclosure provides a Hyaluronic acid-Alginate (HA-Alg) hydrogel comprising a amine-hyaluronic acid (HA-NH₂) solution of a first predefined concentration and an aldehyde-alginate (Alg-CHO) solution of a second predefined concentration, wherein the HA-NH₂ solution and the Alg-CHO solution are mixed in a predefined ratio, and wherein the HA-NH₂ solution comprises HA-NH₂ and a first buffer solution, and the Alg-CHO solution comprises Alg-CHO and a second buffer solution.

Embodiments of the present disclosure substantially eliminate or at least partially address the aforementioned problems in the prior art, and enable cross-linking of the HA-NH₂ and the Alg-CHO to form the HA-Alg hydrogel, which can be effectively used to encapsulate cells, macromolecules, growth factors and/or proteins for various applications.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a flowchart depicting steps of a method for preparing a Hyaluronic acid-Alginate (HA-Alg) hydrogel, in accordance with an embodiment of the present disclosure;

FIG. 2 is a flowchart depicting process steps for synthesizing amine-hyaluronic acid (HA-NH₂), in accordance with an embodiment of the present disclosure;

FIG. 3 is a flowchart depicting process steps for synthesizing aldehyde-alginate (Alg-CHO), in accordance with an embodiment of the present disclosure;

FIG. 4 is an exemplary illustration of a Hyaluronic acid-Alginate (HA-Alg) hydrogel at various predefined ratios of Hyaluronic acid-Alginate (HA-NH₂) solution and aldehyde-alginate (Alg-CHO) solution, in accordance with an embodiment of the present disclosure;

FIG. 5A is an exemplary illustration of a three-dimensional (3D) construct formed by bioprinting using Hyaluronic acid-Alginate (HA-Alg) hydrogel based bioink, in accordance with an embodiment of the present disclosure;

FIG. 5B is an exemplary illustration of cells viability in the Hyaluronic acid-Alginate (HA-Alg) hydrogel based bioink, in accordance with an embodiment of the present disclosure;

FIG. 6 is a graphical representation of NMR spectra of amine-hyaluronic acid (HA-NH₂), and aldehyde-alginate (Alg-CHO) in accordance with an embodiment of the present disclosure;

FIG. 7 are exemplary illustrations of molecular structure of Alginate (Alg), aldehyde-alginate (Alg-CHO) and Hemiacetal, in accordance with an embodiment of the present disclosure;

FIG. 8 is a graphical representation of 1H NMR spectra of Alginate (Alg) and aldehyde-alginate (Alg-CHO), in accordance with an embodiment of the present disclosure;

FIG. 9 is a graphical representation of 13C NMR spectra of Alginate (Alg) and aldehyde-alginate (Alg-CHO), in accordance with an embodiment of the present disclosure;

FIG. 10A is a graphical representation of FTIR spectra of Hyaluronic acid (HA), amine-hyaluronic acid (HA-NH₂), Alginate (Alg) and aldehyde-alginate (Alg-CHO), in accordance with an embodiment of the present disclosure;

FIG. 10B is a graphical representation of FTIR spectra of Hyaluronic acid (HA), Hyaluronic acid-Alginate (HA-Alg) and Silk fibroin (SF), in accordance with an embodiment of the present disclosure;

FIG. 11 is exemplary illustration of molecular structure of Hyaluronic acid-Alginate (HA-Alg) hydrogel, in accordance with an embodiment of the present disclosure;

FIG. 12 is an exemplary illustration of a bioprinter, in accordance with an embodiment of the present disclosure;

FIG. 13 is an exemplary illustration of distribution of fluorescent microspheres, in accordance with an embodiment of the present disclosure;

FIG. 14A is an exemplary illustration of a three-dimensional (3D) construct formed by bioprinting using Hyaluronic acid-Alginate (HA-Alg) hydrogel encapsulated with Silk fibroin (SF) and cells, in accordance with an embodiment of the present disclosure;

FIG. 14B is an exemplary representation of cells viability in the Hyaluronic acid-Alginate (HA-Alg) hydrogel, in accordance with an embodiment of the present disclosure;

FIG. 14C is an exemplary illustration of a construct formed by Hyaluronic acid-Alginate(HA-Alg) hydrogel, in accordance with an embodiment of the present disclosure;

FIG. 15A-15B are exemplary illustrations of distribution of human mesenchymal stem cells (hMSC) in the Hyaluronic acid-Alginate (HA-Alg) hydrogel and the HA-Alg hydrogel encapsulated with Silk-fibroin (SF), respectively, in accordance with an embodiment of the present disclosure;

FIG. 15C is an exemplary illustration of immunochemistry of Ki-67 protein in Hyaluronic acid-Alginate (HA-Alg) hydrogel and the HA-Alg hydrogel encapsulated with Silk-fibroin (SF), in accordance with an embodiment of the present disclosure;

FIG. 15D is an exemplary illustration of staining of glycosaminoglycans (GAGs) secretion in the Hyaluronic acid-Alginate (HA-Alg) hydrogel based bioink and the HA-Alg hydrogel encapsulated with Silk fibroin (SF), in accordance with an embodiment of the present disclosure;

FIG. 16 is a graphical representation of DNA content of Hyaluronic acid-Alginate (HA-Alg) hydrogel, HA-Alg hydrogel encapsulated with Silk fibroin (SF) and fibrin hydrogel, in accordance with an embodiment of the present disclosure;

FIG. 17 is a graphical representation of fluorescence of Hyaluronic acid-Alginate (HA-Alg) hydrogel, HA-Alg hydrogel encapsulated with Silk fibroin (SF) and fibrin hydrogel, in accordance with an embodiment of the present disclosure;

FIG. 18 is an exemplary illustration of compressive loading of the Hyaluronic acid-Alginate (HA-Alg) hydrogel, in accordance with an embodiment of the present disclosure;

FIG. 19 is a graphical representation of variation of compressive strength with respect to strain of Hyaluronic acid-Alginate (HA-Alg) hydrogel and HA-Alg hydrogel encapsulated with Silk fibroin (SF), in accordance with an embodiment of the present disclosure;

FIG. 20 is a graphical representation of elastic modulus of Hyaluronic acid-Alginate (HA-Alg) hydrogel and HA-Alg hydrogel encapsulated with Silk fibroin (SF), in accordance with an embodiment of the present disclosure;

FIG. 21 is a graphical representation of variation of swelling profile and degradation profile of Hyaluronic acid-Alginate (HA-Alg) hydrogel at different predefined ratios of Hyaluronic acid-Alginate (HA-NH₂) solution and aldehyde-alginate (Alg-CHO) solution and HA-Alg hydrogel encapsulated with Silk fibroin (SF), in accordance with an embodiment of the present disclosure;

FIG. 22A is a graphical representation of variation of elastic modulus (G′) and viscous modulus (G″) of Hyaluronic acid-Alginate (HA-Alg) hydrogel and the HA-Alg hydrogel encapsulated with Silk fibroin (SF) with respect to strain, in accordance with an embodiment of the present disclosure;

FIG. 22B is a graphical representation of variation of elastic modulus (G′) and viscous modulus (G″) of Hyaluronic acid-Alginate (HA-Alg) hydrogel and the HA-Alg hydrogel encapsulated with Silk fibroin (SF) with respect to frequency, in accordance with an embodiment of the present disclosure;

FIG. 22C is a graphical representation of variation of elastic modulus (G′) and viscous modulus (G″) of Hyaluronic acid-Alginate (HA-Alg) hydrogel and the HA-Alg hydrogel encapsulated with Silk fibroin (SF) with respect to time, in accordance with an embodiment of the present disclosure;

FIG. 22D is a graphical representation of variation of viscosity of Hyaluronic acid-Alginate (HA-Alg) hydrogel and the HA-Alg hydrogel encapsulated with Silk fibroin (SF) with respect to shear rate, in accordance with an embodiment of the present disclosure; and

FIG. 23 are exemplary illustrations of Immunofluorescent staining of type II collagen in Hyaluronic acid-Alginate (HA-Alg) and the HA-Alg hydrogel encapsulated with Silk fibroin (SF) after two weeks and four weeks, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a method of preparing a Hyaluronic acid-Alginate (HA-Alg) hydrogel, the method comprising:
dissolving a required amount of amine-hyaluronic acid (HA-NH₂) in a first buffer solution to form an HA-NH₂ solution of a first predefined concentration;
dissolving a required amount of aldehyde-alginate (Alg-CHO) in a second buffer solution to form an Alg-CHO solution of a second predefined concentration; and
mixing the HA-NH₂ solution and the Alg-CHO solution in a predefined ratio for preparing the HA-Alg hydrogel.

In another aspect, an embodiment of the present disclosure provides a Hyaluronic acid-Alginate (HA-Alg) hydrogel comprising a amine-hyaluronic acid (HA-NH₂) solution of a first predefined concentration and an aldehyde-alginate (Alg-CHO) solution of a second predefined concentration, wherein the HA-NH₂ solution and the Alg-CHO solution are mixed in a predefined ratio, and wherein the HA-NH₂ solution comprises HA-NH₂ and a first buffer solution, and the Alg-CHO solution comprises Alg-CHO and a second buffer solution.

The present disclosure provides the aforementioned method of preparing the Hyaluronic acid-Alginate (HA-Alg) hydrogel. Mixing the HA-NH₂ solution and the Alg-CHO solution in the predefined concentration results in crosslinking of HA-NH₂ and the Alg-CHO leading to formation of the HA-Alg hydrogel. The HA-Alg hydrogel shows a great potential for encapsulation of different cells leading to synthesis of the HA-Alg hydrogel based bioink. The HA-Alg hydrogel based bioink can be efficiently used in bioprinting, for manufacturing three-dimensional (3D) printed tissues to be used in the regenerative tissue engineering. Further, the HA-Alg hydrogel is non-toxic and therefore, maintains viability of the cells, and supports chondrogenic differentiation of the cells which is an essential requirement to use it as an encapsulating material for the cells. Moreover, the HA-Alg hydrogel exhibits shear thinning properties which is an essential factor for effective printing of the HA-Alg hydrogel.

Additionally, the HA-Alg hydrogel exhibits mechanical properties such as enhanced strength and stability under compressive force which makes it a suitable material for encapsulating macromolecules. Further, the HA-Alg hydrogel exhibits fast gelation. For example, for the HA-Alg hydrogel having the predefined ratio of the HA-NH₂ solution and the Alg-CHO solution of 5:5, gelation may occur within 2 minutes. Moreover, the predefined concentration of the HA-NH₂ solution and the Alg-CHO solution can be manipulated to obtain a required viscosity of the HA-Alg Hydrogel. The viscosity is a crucial factor to enable printability of the HA-Alg hydrogel.

The term “hydrogel” refers to a three-dimensional (3D) crosslinked polymer network, which can retain and absorb large amount of water, while maintaining their structure due to chemical and/or physical cross-linking of individual polymer chains. Herein, the HA-Alg hydrogel is formed by crosslinking the HA-NH₂ and the Alg-CHO.

Notably, the HA-NH₂ solution is formed by dissolving the required amount of the HA-NH₂ in a first buffer solution at the room temperature. Optionally, the HA-NH₂ in dried form is dissolved in the first buffer solution. The required amount of the HA-NH₂ could lie in a range of 10 mg/ml-30 mg/ml. For example, the required amount of the HA-NH₂ lies in a range of 10 mg/ml, 15 mg/ml, or 25 mg/ml up to 12 mg/ml, 24 mg/ml, or 30 mg/ml. Optionally, the HA-NH₂ and the first buffer solution are filtered before use. Optionally, the HA-NH₂ is dissolved in the first buffer solution in a biosafety cabinet.

Similarly, Alg-CHO solution is formed by dissolving the required amount of the Alg-CHO in the second buffer solution at the room temperature. Optionally, the Alg-CHO in the dried form is dissolved in the second buffer solution. The required amount of the Alg-CHO could lie in a range of 10 mg/ml-30 mg/ml. For example, the required amount of the HA-NH₂ lies in a range of 10 mg/ml, 15 mg/ml, or 25 mg/ml up to 12 mg/ml, 24 mg/ml, or 30 mg/ml. Optionally, the Alg-CHO and the second buffer solution are filtered before use. Optionally, the Alg-CHO is dissolved in the second buffer solution in the biosafety cabinet.

Optionally, the first predefined concentration lies in a range of 10-30 mg/ml and the second predefined concentration lies in a range of 10-20 mg/ml. The first predefined concentration and the second predefined concentration is crucial for triggering gelation to form the HA-Alg hydrogel. As an example, the first predefined concentration lies in a range of 10 mg/ml, 15 mg/ml, or 25 mg/ml up to 12 mg/ml, 24 mg/ml, or 30 m/ml. As another example, the second predefined concentration lies in a range of 10 mg/ml, 12 mg/ml, 14 mg/ml, or 17 mg/ml up to 11 mg/ml, 15 mg/ml, 18 mg/ml, or 20 mg·ml. In one implementation, the first predefined concentration may be 30 mg/ml. In another implementation, the second predefined concentration is 20 mg/ml. Advantageously, the first predefined concentration and the second predefined concentration imparts optimum viscosity to the HA-NH₂ solution and the Alg-CHO solution, respectively resulting in handling of the aforesaid solutions with ease.

Optionally, the first buffer solution and the second buffer solution is implemented as a Phosphate buffered saline (PBS) solution. In this regard, the PBS is buffered salt solution maintained at a pH of approximately equal to 7.4. The PBS is a water-based salt solution comprising each of disodium hydrogen phosphate (Na₂HPO₄), sodium chloride (NaCl), potassium chloride (KCl), potassium dihydrogen phosphate (KH₂PO₄) in predefined concentrations. Optionally, the PBS solution is 1×PBS solution. In this regard, the 1×PBS solution contains 8.0 gram/Liter of NaCl, 0.2 gram/Liter of KCl, 1.42 gram/Liter of Na₂HPO₄ and 0.24 gram/Liter of KH₂PO₄.

Advantageously, the PBS solution maintains a constant pH level, is stable and non-toxic, hence does not interfere with chemical properties of the HA-NH₂ and the Alg-CHO resulting in effective crosslinking to form the HA-Alg hydrogel.

Notably, upon forming the HA-NH₂ solution and the Alg-CHO solution, the aforesaid solutions are mixed together to facilitate crosslinking between the HA-NH₂ and the Alg-CHO. Crosslinking between the HA-NH₂ and Alg-CHO resulted by formation of covalent bonds between amine group (NH₂) on the Hyaluronic acid (HA) and aldehyde group (CHO) on Alg, known as Schiffs's base reaction. In particular, covalent bonds are formed between one carbon atom of the CHO and one Nitrogen atom of the NH₂ to form an imine bond. Optionally, gelation is initiated to form the HA-Alg hydrogel through the Schiff s base reaction. Optionally, the HA-NH₂ and the Alg-CHO solutions are mixed in the biosafety cabinet at the room temperature. Optionally, the predefined ratio of the HA-NH₂ solution and the Alg-CHO solution is one of: 5:5, 6:4, 7:3 respectively. In this regard, the aforesaid solutions are mixed in different ratios to trigger the gelation to from the HA-Alg hydrogel. Optionally, the HA-Alg hydrogel have different properties depending upon the predefined concentration of the HA-NH₂ and the Alg-CHO solution. Properties of the HA-Alg hydrogel may include, gelation time, degradation, injectability through needles, and the like. As one example, the gelation time may increase upon increasing concentration of the HA-NH₂ solution. Herein, upon mixing the HA-NH₂ solution and the Alg-CHO solution in the predefined ratio of 5:5 respectively, the gelation may take place in 1 minute-3 minutes. For example, the gelation takes place in 1 minute, 1.5 minute, 2 minute or 2.5 minute up to 1.2 minute, 2 minute, 2.5 minute or 3 minute. Upon mixing the HA-NH₂ solution and the Alg-CHO solution in the predefined ratio of 6:4 respectively, the gelation may take place in 2 minutes-4 minutes. For example, the gelation takes place in 2 minutes, 2.5 minutes, 3 minutes or 3.5 minutes up to, 2.2 minutes, 3 minutes, 3.5 minutes or 4 minutes. Upon mixing the HA-NH₂ solution and the Alg-CHO solution in the predefined ratio of 7:3 respectively, the gelation may take place in 3 minutes-5 minutes. For example, the gelation takes place in 3 minutes, 3.5 minutes, or 4 minutes up to, 3.2 minutes, 4.2 minutes, or 5 minutes.

In one implementation, 100 microliter (μL) of the HA-NH₂ solution and 100 μL of the Alg-CHO solution may be mixed. In said implementation, the gelation may take place in approximately 2 minutes. In another implementation, 120 μL of the HA-NH₂ solution and 80 μL of the Alg-CHO solution may be mixed. In said implementation, the gelation may take place in approximately 3 minutes. In yet another implementation, 140 μL of the HA-NH₂ solution and the 60 μL of the Alg-CHO solution may be mixed. In said implementation, the gelation may take place in approximately 4 minutes.

Further, the predefined concentration impacts the degradation of the HA-Alg hydrogel. Herein, the HA-Alg hydrogel, having a high concentration of the HA-NH₂ reduces rigidity, resulting in degradation of the HA-Alg hydrogel. Upon increasing the concentration of the HA-NH₂, the HA-Alg hydrogel absorbs more water due to interaction between hydroxyl groups on its backbone and water molecules. Therefore, molecular structure of the HA-NH₂ stretches out, water content in the HA-Alg hydrogel increases, and resulting in generation of high swelling pressure. The high swelling pressure reduces rigidity of the HA-Alg hydrogel, which induces degradation of HA-Alg hydrogel. The swelling pressure and degradation behaviors of the HA-Alg Hydrogel at different predefined ratio of the HA-NH₂ and Alg-CHO is described in experimental part. It will be appreciated that, other values of the predefined ratio of the HA-NH₂ solution and the Alg-CHO solution are also feasible. Advantageously, the technical effect of different predefined ratios of the HA-NH₂ solution and the Alg-CHO solution is that the HA-Alg hydrogel may be tuned for varying application, resulting in significant enhancement in usability of the HA-Alg hydrogel.

Optionally, the method further comprises modifying Hyaluronic acid (HA) for synthesizing the HA-NH₂, the method comprising:
dissolving the HA in deionized water (DI) for preparing a HA solution of a third predefined concentration;
adding a required amount of ethylene-diamine in the HA solution;
adjusting pH of the HA solution to form a HA reaction mixture having a required pH;
dissolving a required amount of 1-ethyl-3-(3-dimethylaminopropyl) carbodiimide hydrochloride (EDC) and a required amount of 1-hydroxybenzotriazole hydrate (HOBt) in a third buffer solution for preparing an EDC-HOBt solution;
adding the EDC-HOBt solution to the HA reaction mixture for obtaining a modified HA reaction mixture; and
dialyzing the modified HA reaction mixture with the DI water for obtaining the HA-NH₂.

In this regard, the HA is chemically modified to conjugate amine group (NH₂) on its polymeric structure. Notably, a required amount of the HA is dissolved in a required volume of the DI water to prepare the HA solution of the third predefined concentration. The required amount of the HA could lie in a range of 2.5 mg/ml-10 mg/ml. For example, the required amount of the HA lies in a range of 2.5 mg/ml, 3.5 mg/ml, 5.5 mg/ml, or 7.5 mg/ml up to 5 mg/ml, 7 mg/ml, 8.5 mg/ml or 10 mg/ml. The required volume of the DI water could lie in a range of 0.5 ml to 2 ml. As an example, 0.5 grams of the HA may be dissolved in 100 ml of the DI water to obtain the third predefined concentration of 5 mg/ml. Optionally, the required amount of the ethylene-diamine could lie in a range of 50 nanomoles (nmol) to 100 nmol. For example, the range may be 50 nmol, 60 nmol, 70 nmol, or 85 nmol up to 75 nmol, 85 nmol, 95 nmol, or 100 nmol. In one implementation, 94 nmol of the ethylene-diamine may be added in the HA solution. Optionally, the pH of the HA solution is adjusted by adding a required amount of acid in the HA solution. The acid could be one of: Hydrochloric acid (HCl), phosphoric acid, nitric acid, sulfuric acid. In an example, the HCl may be added. The acid may be added in a drop wise manner until a required pH of the HA reaction mixture is achieved.

Further, in the method, the EDC-HOBt solution is prepared by dissolving a required amount of the EDC and a required amount of the HOBt in a required volume of the third buffer solution. Optionally, the third buffer solution is at least one of: PBS buffer solution and Dimethyl sulfoxide (DMSO) solution. Optionally, the required volume of the third buffer solution could be in a range of 10 ml-12 ml. As an example, 0.8 g of the EDC and 0.7 g of the HOBt may be dissolved in 10 ml of the DMSO solution. Optionally, 10 ml of the the EDC-HOBt solution is mixed in the HA reaction mixture in a drop wise manner. Herein, the carboxyl groups (—COOH) on the HA are activated by the EDC, resulting in attachment of the NH₂ on the HA. The HOBt stabilizes HA-NH₂ molecules in the HA reaction mixture.

Optionally, the modified HA reaction mixture is stirred for twenty-four hours at the room temperature. Further, in the method, the modified HA reaction mixture is dialyzed with the DI water for a required time duration. The required time duration could lie in a range of 2 days-5 days. As an example, the required time duration is 3 days. Optionally, the DI water is changed for every 8 hours to avoid contamination of the HA reaction mixture. Optionally, the step of dialyzing the modified HA reaction mixture is performed using a dialysis bag, the dialysis bag has a plurality of pores having a pore size lying in a range of 11,000 Dalton to 15,000 Dalton. For example, the pore size may lie in a range of 11,000 Da, 11,500 Da, 12,000 Da, or 13,000 Da up to 12,000 Da, 14,000 Da, or 14,500 Da, or 15,000 Da. In an implementation, the pore size may be 14,000 Da. Advantageously, the aforesaid pore size of the dialysis bag allows passage of molecules having size smaller than 14,000 Da.

Optionally, upon dialysis, the HA reaction mixture from the dialysis bag is transferred into a sterile container and subjected to salting out by addition of a salt to a concentration of 5% w/v. Addition of the salt results in formation of a HA-NH₂ precipitate. Optionally, the HA-NH₂ precipitate is collected in 70% ethanol. Additionally, optionally, the HA-NH₂ precipitate is dissolved in water, and dialyzed for 3 days to remove the salt. Removal of the salt results in synthesis of the HA-NH₂. Optionally, the HA-NH₂ is freeze-dried and stored at 4 degrees Celsius. Optionally, the HA-NH₂ is subjected to UV sterilization for approximately 20 minutes in the biosafety cabinet before use.

Optionally, the method further comprises modifying Alginate (Alg) for synthesizing the Alg-CHO, the method comprising:
dissolving the Alg in deionized (DI) water for preparing an Alg solution of a fourth predefined concentration;
adding a required amount of Sodium periodate (NaIO₄) to the Alg solution for obtaining a modified Alg solution; and
dialyzing the modified Alg solution with the DI water for obtaining the Alg-CHO.

In this regard, the Alg is chemically modified to conjugate the Aldehyde group (CHO) on its polymeric structure. Notably, a required amount of the Alg is dissolved in a required volume of the DI water to prepare the Alg solution of the fourth predefined concentration. The required amount of the Alg could lie in a range of 1% w/v-4% w/v. For example, the required amount lies in a range of 1% w/v, 1.5% w/v, 2% w/v, or 2.5% w/v up to 1.2% w/v, 2.2% w/v, 3% w/v or 4% w/v. The required volume of the DI water could lie in a range of 100 ml-200 ml. For example, the required volume lies in a range of 100 ml, 110 ml, 130 ml, or 160 ml up to 120 ml, 160 ml, 180 ml, or 200 ml. In one implementation, 2 grams of the Alg may be dissolved in 100 ml of the DI water to obtain the Alg solution of the fourth predefined concentration of 2 mg/ml.

Optionally, the required amount of the NaIO₄ could be in a range of 4 ml to 6 ml. As an example, 5 ml of the NaIO₄ is added in the Alg solution. Optionally, upon adding the NaIO₄ solution, the Alg solution is stirred at the room temperature for two hours in dark. Optionally, a condition of the dark is obtained by wrapping a container having the Alg solution with an Aluminum foil. The dark condition is obtained to prevent excess light which can interfere with reaction between the Alg-CHO solution and the NaIO₄. Optionally, a required amount of Ethylene glycol is used to quench excess NaIO₄ solution in the Alg solution.

Further, in the method, the Alg solution is dialyzed with the DI water for a required time duration. The required time duration could lie in a range of 2 days-5 days. As an example, the required time duration is 3 days. Optionally, the DI water is changed for every 8 hours to avoid contamination of the Alg solution. Optionally, the step of dialyzing the modified Alg solution is performed using a dialysis bag, the dialysis bag has a plurality of pores having a pore size lying in a range of 11,000 Dalton (Da) to 15,000 Dalton. In this regard, the pore size of the dialysis bag is a crucial factor to facilitate effective separation of molecules present in the Alg solution. Optionally, the molecules having a size greater than the pore size cannot pass through the dialysis bag. For example, the pore size lie in a range of 11,000 Da, 11,500 Da, 12,000 Da, or 13,000 Da up to 12,000 Da, 14,000 Da, or 14,500 Da, or 15,000 Da. Advantageously, the aforesaid pore size allows removal of particles less than a size of 14000 Da from the Alg solution.

Optionally, upon dialysis, the Alg solution from the dialysis bag is transferred into a sterile container and subjected to salting out by addition of a salt to a concentration of 5% w/v. Addition of the salt results in formation of a Alg-CHO precipitate. Optionally, the Alg-CHO precipitate is collected in 70% ethanol. Additionally, optionally, the Alg-CHO precipitate is dissolved in water, and dialyzed for 3 days to remove the salt. Remove of the salt results in synthesis of the Alg-CHO. Optionally, the Alg-CHO is freeze-dried and stored at 4 degrees Celsius. Optionally, the Alg-CHO is subjected to UV sterilization for approximately 20 minutes in the biosafety cabinet before use. Optionally, chemical modification of the HA and the Alg is confirmed by Nuclear Magnetic Resonance (NMR) and Fourier transform infrared spectroscopy (FTIR).

Optionally, the step of mixing the HA-NH₂ solution and the Alg-CHO solution comprises:
mixing a predefined concentration of cells in a required volume of the Alg-CHO solution to form a cell mixture; and
mixing an equal volume of the HA-NH₂NH₂ solution with the cell mixture for synthesizing a HA-Alg hydrogel based bioink, wherein the HA-Alg hydrogel based bioink is usable in a bioprinter for bioprinting.

In this regard, the cells are mixed in the Alg-CHO solution and the HA-NH₂ solution to form the HA-Alg hydrogel based bioink. Optionally, the HA-Alg hydrogel based bioink is used for a purpose of 3-dimensional (3D) bioprinting of biological materials using the bioprinter. The biological materials could be at least one of: tissues, organs, peptides, proteins, polysaccharides. 3D bioprinting techniques could be at least one of: extrusion-based bioprinting, injek-based bioprinting, laser-assisted bioprinting, stereolithography. In one implementation, the extrusion-based 3D bioprinting may be used. The Extrusion-based 3D bioprinting involves dispensation of the HA-Alg hydrogel based bioink from a nozzle extruder of the bioprinter incorporated with x, y, z motion system. The bioprinter may be a conventional a bioprinter, known in the art. The HA-Alg hydrogel based bioink contains living cells and biomaterials to be differentiated into the biological material. HA-Alg hydrogel based bioink mimics extracellular matrix environment, supports cell adhesion, proliferation, and differentiation after printing.

Optionally, a type of cells to be mixed in the Alg-CHO solution and the HA-NH₂ solution may depend upon application of the HA-Alg hydrogel based bioink. Optionally, the cells could be at least one of: Human stromal (mesenchymal) stem cells (hMSC), induced pluripotent stem cells (iPSC), Keratinocytes, Fibroblasts, Muscle Derived Stem Cells, Neural Stem Cells, Neonatal Cardiomyocytes. As an example, the hMSC may be used to form the HA-Alg hydrogel based bioink. The predefined concentration of the cells could lie in a range of 10⁵-10⁶ cells/ml. As an example, hMSC cells having the predefined concentration of 10⁶ cells/mL may be used to form the bioink. Optionally, the cells are cultured in a media to obtain the predefined concentration.

Optionally, the media could be at least one of: serum containing media, serum free media, chemically defined media, protein-free media. As an example, the hMSC may be maintained in the media supplemented with 5% (v/v) Fetal Bovine Serum (FBS), 1% (v/v) GlutaMAX, 1% (v/v), 4-(2-hydroxyethyl)-1-piperazineethanesulfonic acid (HEPES) buffer, 1 ng/mL of fibroblast growth factor-basic (bFGF) (Invitrogen), and 1% (v/v) antibiotic-antimycotic solution at 37 degrees Celsius, and 5% CO2. Optionally, the media is changed every 3 days. Optionally, subculturing is performed using when the cells reached 80% confluency.

Notably, upon achieving the predefined concentration of the cells, the cells are mixed in the required volume of the Alg-CHO solution to form the cell mixture. Optionally, the required volume of the Alg-CHO solution could lie in a range of 10 mg/ml-30 mg/ml. Optionally, the cells are mixed in the Alg-CHO solution at the room temperature in a Laminal air flow. Further, in the method, the equal volume of the HA-NH₂ solution is added to the cell mixture resulting in crosslinking of the carbon in the Alg-CHO solution and the nitrogen in the HA-NH₂ solution. Optionally, crosslinking leads to encapsulation of the cells in the HA-Alg hydrogel resulting in synthesis of the HA-Alg hydrogel based bioink. Advantageously, the HA-Alg hydrogel based bioink can be easily passed through nozzles of the bioprinter to enable in printing and supports Chondrogenic differentiation of the cells into different tissues.

Optionally, the step of mixing the HA-NH₂ solution and the Alg-CHO solution comprises:
adding a required volume of the HA-NH₂ solution to a required volume of macromolecule solution for synthesizing a HA-NH₂ macromolecule solution; and
adding an equal volume of the Alg-CHO solution to the HA-NH₂ macromolecule solution for encapsulating macromolecules in the HA-Alg hydrogel.

In this regard, the HA-Alg hydrogel is utilized as an interpenetrating polymer network (IPN) platform to encapsulate the macromolecules. Optionally, the macromolecules are silk fibroins (SF). Other example of the macromolecules could be, but are not limited to, collagen, bacterial cellulose. Optionally, the required concentration of the macromolecule solution lies in a range of 1 mg/ml-4 mg/ml. For example, the required concentration of the macromolecule solution lies in a range of 1 mg/ml, 1.5 mg/ml, 2 mg/ml, or 2.5 mg/ml up to 1.2 mg/ml, 2.2 mg/ml, 3 mg/ml, or 4 mg/ml. The required concentration of the HA-NH₂ solution lies in a range of 10 mg/ml-40 mg/ml. For example, the required concentration of the HA-NH₂ solution lies in a range of 10 mg/ml, 15 mg/ml, 20 mg/ml, or 30 mg/ml up to 15 mg/ml, 25 mg/ml, 35 mg/ml, or 40 mg/ml. Optionally, the step of adding the macromolecule solution in the HA-NH₂ solution is performed at the room temperature. Further, in the method, addition of the equal volume of the Alg-CHO solution to the macromolecule solution results in crosslinking of the carbon atom of the Alg-CHO solution with the nitrogen atom of the HA-NH₂ solution resulting in encapsulation of the macromolecules in the HA-Alg hydrogel.

As an example, the HA-Alg hydrogel may be utilized to encapsulate silk fibroin (SF) macromolecules. To encapsulate the SF macromolecules, a SF solution is prepared. With regard to this, 4 g of degummed silk is dissolved in 16 ml of 9.3 M Lithium bromide (LiBr) at a temperature of 60 degree Celsius for a time duration of 4 hours to form a SF solution. Further, the SF solution is dialyzed with the DI water for 3 days to form the dialyzed SF solution. Optionally, the DI water is changed every 8 hours. Further, the dialyzed SF solution is centrifuged at a speed of 9000 rotations per minute (rpm), at the temperature of 4 degree Celsius for the time duration of 20 minutes. Optionally, the SF solution is centrifuged two times. Lastly, the SF solution is adjusted to achieve a concentration of 6 mg/ml in the DI water.

Further, the SF solution may be added to 20 mg/ml of the HA-NH₂ solution to obtain a concentration of 30 mg/mL of the HA-NH₂ and 2 mg/mL of the SF, resulting in synthesis of a HA-NH₂ SF solution. Lastly, the equal volume of the Alg-CHO solution may be added to the HA-NH₂ SF solution to encapsulate the SF macromolecules in the HA-Alg hydrogel. Advantageously, the technical effect of using the aforesaid method is that the macromolecules can be easily encapsulated in the HA-Alg hydrogel resulting in significant enhancement in usability of the HA-Alg hydrogel.

Optionally, the step of mixing the HA-NH₂ solution and the Alg-CHO solution comprises:
adding a required concentration of growth factor and/or protein to a required volume of the Alg-CHO solution to form a growth factor and/or protein solution; and
adding an equal volume of the HA-NH₂ solution to the growth factor and/or protein solution for encapsulating growth factors and/or proteins in the HA-Alg hydrogel, wherein the growth factors and/or proteins are released controllably.

In this regard, the growth factors and/or proteins are mixed in the Alg-CHO and the HA-NH₂ solution to encapsulate the growth factors and/or proteins and to enable in controlled release of the same. Optionally, the required concentration of the growth factors could lie in a range of 10-20 nanogram/ml (ng/ml). For example, required concentration of the growth factors may lie in range of 10 ng/ml, 12 ng/ml, or 15 ng/ml up to 13 ng/ml, 16 ng/ml, 18 ng/ml, or 20 ng/ml. In one implementation, the required concentration of the growth factors is 10 ng/ml. Optionally, the required concentration of the proteins could lie in a range of 10-50 mg/ml. For example, the required concentration of the proteins lies in a range of 10 mg/ml, 15 mg/ml, 25 mg/ml, or 35 mg/ml up to, 20 mg/ml, 30 mg/ml, 40 mg/ml, 45 mg/ml, or 50 mg/ml. In one implementation, the required concentration of the proteins is 40 mg/ml. Optionally, the growth factor and/or protein is added to the Alg-CHO solution in the biosafety cabinet at the room temperature.

Further, in the method, the equal volume of the HA-NH₂ is added in the growth factor/protein solution. Optionally, the first predefined concentration of the HA-NH₂ solution is 18 mg/ml. Addition of the equal volume of the HA-NH₂ solution to the growth factor and/or protein solution results in crosslinking of the carbon atom of the Alg-CHO solution with the nitrogen atom of the HA-NH₂ solution resulting in encapsulation of the growth factor and/or protein in the HA-Alg hydrogel. Optionally, the HA-Alg hydrogel slowly starts to degrade, resulting in release of the growth factors and/or proteins encapsulated in the HA-Alg hydrogel. In one implementation, the Alg-CHO, HA-NH₂, and the proteins may be mixed with each other in a ratio of 5:4:1. In said implementation, 6.2% of the proteins are released. In another implementation, the Alg-CHO, HA-NH₂, and the proteins may be mixed with each other in the ratio of 6:3:1. In said implementation, 22.7% of the proteins are released.

The present disclosure also relates to the Hyaluronic acid-Alginate (HA-Alg) hydrogel as described above. Various embodiments and variants disclosed above, with respect to the aforementioned first aspect, apply mutatis mutandis to the Hyaluronic acid-Alginate (HA-Alg) hydrogel.

Optionally, the predefined ratio of the HA-NH₂ solution and the Alg-CHO solution is one of: 5:5, 6:4, 7:3 respectively.

Optionally, the HA-Alg hydrogel further comprises a predefined concentration of cells mixed in required volume of the Alg-CHO solution to form a cell mixture, wherein an equal volume of the HA-NH₂ solution mixed with the cell mixture enables synthesis of a HA-Alg hydrogel based bioink, and wherein the HA-Alg hydrogel based bioink is usable in a bioprinter for bioprinting. Optionally, The HA-Alg hydrogel further comprises a required volume of macromolecule solution mixed in a required volume of the HA-NH₂ solution to synthesize a HA-NH₂ macromolecule solution, wherein an equal volume of the Alg-CHO solution mixed with the HA-NH₂ macromolecule solution enables encapsulation of macromolecules in the HA-Alg hydrogel.

Optionally, the HA-Alg hydrogel further comprises a required concentration of growth factors and/or proteins mixed in a required volume of the Alg-CHO solution to form the growth factor and/or protein solution, wherein an equal volume of the HA-NH₂ solution mixed with the growth factor and/or protein solution enables encapsulation of growth factors and/or proteins, wherein the growth factors and/or proteins are released controllably.

Optionally, the HA-Alg hydrogel has a viscosity lying in a range of 30 Pascal-second-32 Pascal-second. In this regard, the viscosity of the HA-Alg hydrogel is determined by Rheological analysis. Optionally, the viscosity of the HA-Alg hydrogel depends on the predefined ratio of the HA-NH₂ and the Alg-CHO. For example, increasing concentration of the HA-NH₂ solution may result in decrease in viscosity of the HA-Alg hydrogel. In one implementation, the viscosity of the HA-Alg hydrogel having the predefined ratio of 5:5 of the HA-NH₂ solution and the Alg-CHO solution, respectively could lie in a range of 30 pascal-second (Pa·s) to 32 Pa·s. As an example, the viscosity may be 30.4 Pa·s.NH₂NH₂. Optionally, the viscosity of the HA-Alg hydrogel is a crucial factor to effectively encapsulate the cells and use the HA-Alg hydrogel based bioink for the purpose of 3D bioprinting using the bioprinter.

### Experimental Data for Characterization of the HA-NH₂ and the Alg-CHO by NMR Analysis

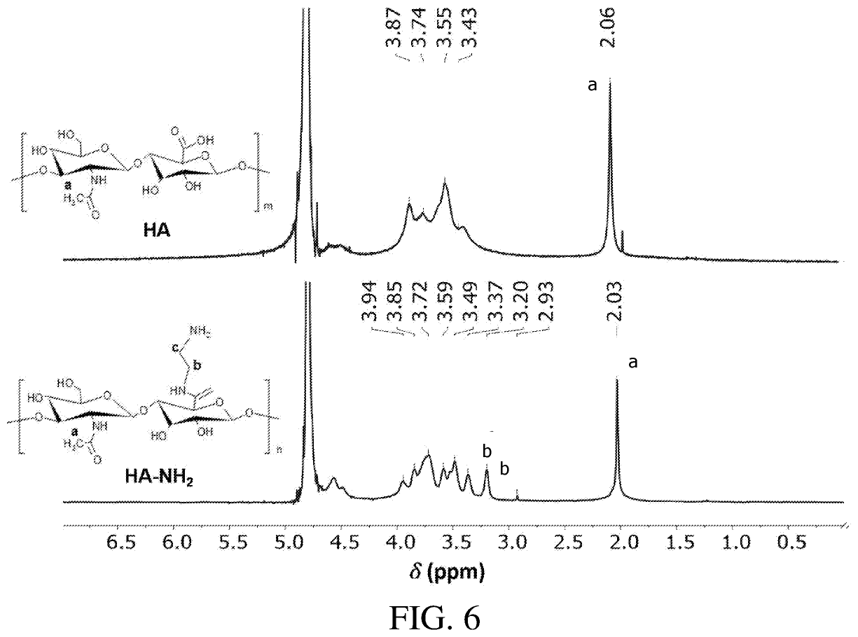

Modification of the HA and the Alg is confirmed by the NMR analysis. In this regard, an unmodified HA, unmodified Alg, freeze-dried HA-NH₂, and freeze-dried Alg-CHO were dissolved in deuterium oxide (D₂O) and transferred to NMR sample tubes of 5 mm length. The NMR spectra of each sample were recorded by a spectrometer. One dimensional 1H and 13C resonances were obtained and analyzed. 1H NMR was performed to characterize HA and confirm the modification of the HA with the NH₂. An NMR spectrum of the HA-NH₂ and the Alg-CHO is depicted in FIG. 6. In the spectrum obtained for the HA, signals from 3.87 to 3.43 ppm were assigned to protons on the sugar rings. Carboxylate groups of the HA were activated and conjugated with primary amines using the ethylenediamine using the EDC-HOBt solution. Methyl (—CH3) protons of N-acetyl group of the HA and the HA-NH₂ showed a signal at 2.06 ppm (FIG. 6, label a). In the spectrum obtained for the HA-NH₂, presence of conjugated ethylenediamine was confirmed by proton signals at 3.20 ppm (FIG. 6, label b) and 2.93 ppm (FIG. 6, label c). The HA-NH₂ also exhibited new proton signals at 3.94 and 3.37 ppm, indicating chemical shift of the protons in sugar rings after conjugating with the primary amines.

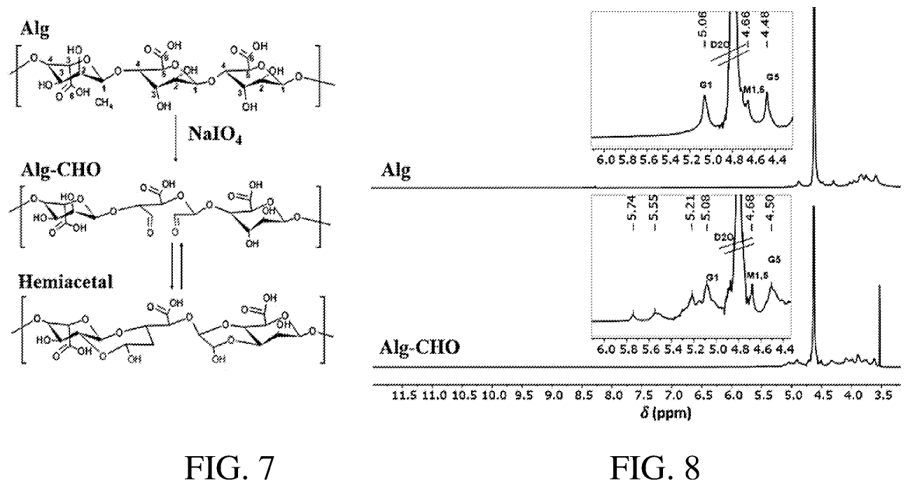

Further, the Alg and the Alg-CHO were characterized by 1H and 13C NMR to confirm the products of oxidation. In this regard, Alg was reacted with the NaIO₄ resulting in the cleavage of the carbon bond between C-2 and C-3, leading to the formation of two aldehyde groups on its polymer chains as indicated by Alg-CHO. Molecular structures of the Alg, Alg-CHO and Hemiacetal is depicted in FIG. 7. The 1H NMR spectra of the Alg and the Alg-CHO exhibited signals at 5.06, 4.66, and 4.48 ppm corresponding to the protons at positions, G1, M1, and G5 on Alg backbone. The 1H NMR spectra of the Alg and the Alg-CHO are depicted in FIG. 8. The peak at 9.3-9.7 ppm, representing aldehyde groups, were not detected on the 1H NMR spectrum of the Alg-CHO. Instead, peaks at 5.74 and 5.55 ppm, which were reported as the signal of hemiacetal after oxidation of the Alg with the NaIO₄, were detected, CHO group on the Alg-CHO reacted with the hydroxyl groups (—OH) on the adjacent sugar rings and formed hemiacetal, which is reversible in aqueous solution.

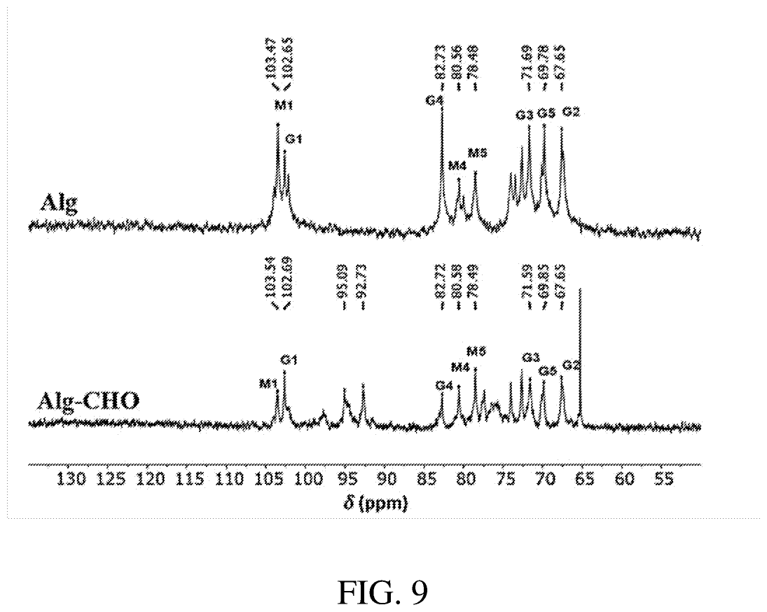

For 13C NMR spectra, both of the Alg and the Alg-CHO showed signals corresponding to G blocks (G1, G2, G3, G4 and G5) and M blocks (M1, M4 and M5) on their sugar rings. The 13C NMR spectrum of the Alg and the Alg-CHO is depicted in FIG. 9. The 13C NMR spectrum of the Alg-CHO had new signals at 95.09 ppm and 92.73 ppm, which were not present in the unmodified Alg. Like, the 1H-NMR spectrum these new signal corresponded to hemiacetalic carbons. Although the signal of the CHO group on the Alg-CHO were not present in the NMR spectra, the signals of the hemiacetalic protons demonstrated successful modification of the Alg to the Alg-CHO.

### Experimental Data for Characterization of the HA-NH₂ and the Alg-CHO by FTIR Analysis

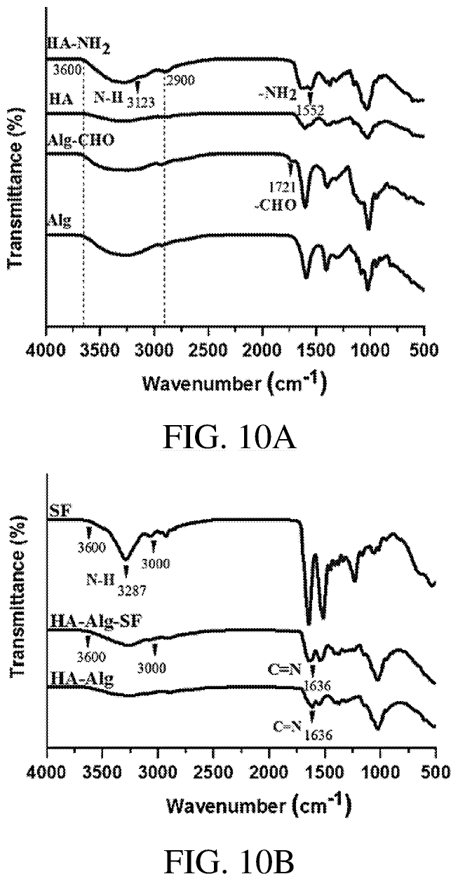

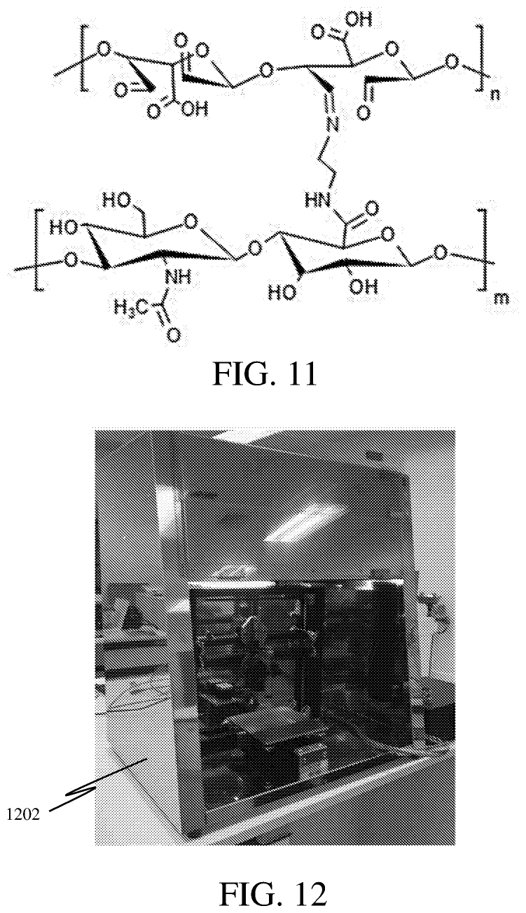

Modification of the HA and the Alg is confirmed by the FTIR analysis. In this regard, the unmodified HA, the unmodified Alg, the freeze-dried HA-NH₂, and the freeze-dried Alg-CHO were mixed with Potassium chloride (KBr) powder. FTIR spectrums were obtained in a range of wavenumber from 4000 cm⁻¹ to 500 cm⁻¹. The FTIR spectrums were averaged over 64 scans with the wavenumber 4.0 cm⁻¹ resolutions. FTIR spectrums of the HA and the Alg were determined before and after modification to confirm addition of the NH₂ and the CHO on the HA and the Alg, respectively. A FTIR spectrum of the HA showed stretching of O—H bond of the hydroxyl groups at 3600-2900 cm⁻¹. In the FTIR spectrum of the HA-NH₂, a new peak at 3123 cm⁻¹ was observed which indicated stretching of N—H bond in amine salt after chemical modification. The NH₂ in the HA-NH₂ was confirmed by peak of 1552 cm⁻¹. Modification of the Alg by the CHO was confirmed by a peak at 1721 cm⁻¹, which corresponded with the NMR analysis. The FTIR spectrum of the HA, the HA-NH₂, the Alg and the Alg-CHO is depicted in FIG. 10A. The FTIR spectrum of HA-Alg, HA-Alg SF and the SF is depicted in FIG. 10B. Gelation of the HA-Alg hydrogel occurred as a result of the formation of the imine bond (C═N) via the Schiff's base reaction between the CHO and the NH₂ groups, as demonstrated by peak at 1636 cm-1. Similarly, the peak at 1636 cm⁻¹ was observed in the HA-Alg hydrogel encapsulated with the SF. Depletion of the CHO at the peak 1721 cm⁻¹ might have occurred as a result of formation of the imine bonds between the CHO on the Alg and the NH₂ on the HA. The molecular structure of the HA-Alg is depicted in FIG. 11.

Encapsulation of the SF into the HA-Alg hydrogel resulted in shifting of the FTIR spectrum in the 3600 cm⁻¹ to 3000 cm⁻¹ region, indicating presence of free NH₂ on the SF which did not react with the Alg-CHO to form the imine bonds (FIG. 10B). The SF contains small amount of primary NH₂ in Lysine which is less than 0.5 mol %, which may have possibly reacted with the CHO on the modified Alg. Therefore, the SF was mixed with the HA-NH₂ before forming hydrogel with the Alg-CHO. In addition, large amount of non-primary NH₂ on the SF cannot induce gelation after mixing with the Alg-CHO. The crosslinking system was mainly based on the Schiff's base reaction between the CHO and primary NH₂ on the Alg-CHO and HA-NH₂, respectively.

Experimental Data for Printability of the HA-Alg Hydrogel and the HA-Alg Hydrogel Encapsulated with the SF

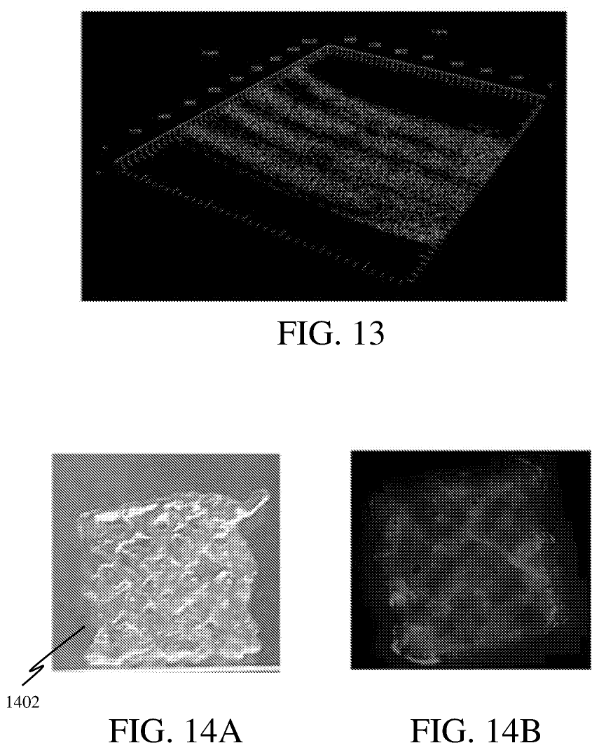

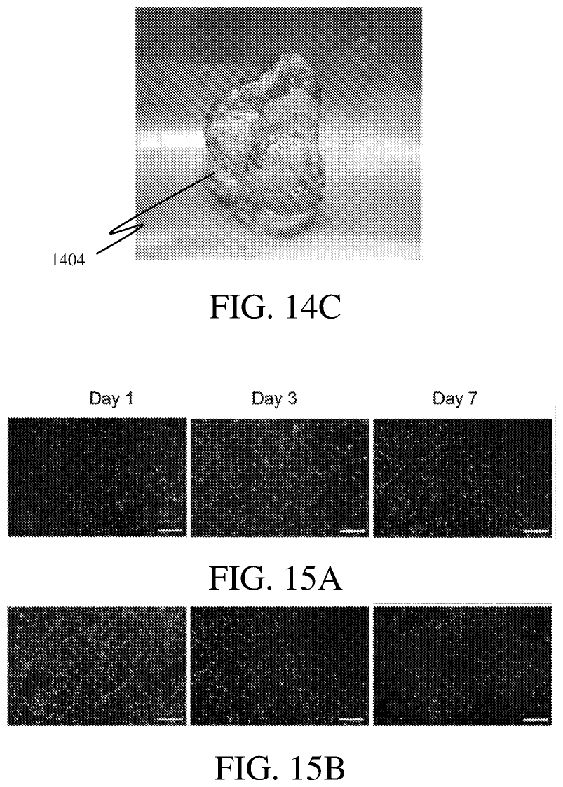

The Alg-CHO was mixed with Polystyrene Microsphere, combined with the equal volume of the HA-NH₂ to form the HA-Alg hydrogel. The HA-Alg hydrogel was transferred into a syringe of volume 1 ml and printed through 18 G needle. An outer diameter of the needled was 1.27 mm, and an inner diameter of the needle was 0.838 mm. The mixture was printed using a custom-made 3D printer equipped with a screw-driven extruder as depicted in FIG. 12. The HA-Alg hydrogel and the HA-Alg hydrogel encapsulated with the SF were printed into a grid pattern of five layers. A length of the of the grid patterns was 20 mm, a width of the grid pattern was 20 mm, and a thickness of the grid pattern was 5 mm. Printing was performed at a temperature of 37 degrees Celsius with a speed of 60 mm/min. The HA-Alg (5:5) and HA-Alg hydrogel encapsulated with the SF were able to print, as demonstrated by define strands and a homogenous distribution of fluorescent microspheres as depicted in FIG. 13. A distribution of the microspheres in the HA-Alg hydrogel was visualized under confocal microscope. Further, construct formed by the encapsulating the SF and the hMSC in the HA-Alg hydrogel is depicted in FIG. 14A. Cell viability of the hMSC in the HA-ALg hydrogel encapsulate with the SF and the hMSC is depicted in FIG. 14B. Further, the HA-Alg hydrogel was printed into an ear-shape structure for reconstructive surgery as depicted in FIG. 14C.

### Experimental Data for Printability of the HA-Alg Hydrogel Based Bioink.

Printability of the HA-Alg hydrogel based bioink was determined to establish its effectivity for 3D bioprinting. Herein, the HA-Alg hydrogel based bioink was transferred into a 1 ml syringe and printed through 18 G needle using a custom-made 3D printer. An outer dimeter of the needle was 1.27 mm and an inner diameter of the needle was 0.838 mm. The HA-Alg hydrogel based bioink was printed to form constructs of two layers having a grid pattern. Dimensions of the constructs were 15 mm×15 mm×0.2 mm. Printing was performed at 37 degree Celsius with a speed of 60 mm/min.

### Experimental Data for Chondrogenic Differentiation of the Cells

Constructs formed using the HA-Alg hydrogel based bioink was cultured in chondrogenic media. The media was glucose-(Dulbecco's Modified Eagle Medium) DMEM media supplemented with 1% (v/v) insulin-transferrin-selenium, 1% (v/v) HEPES buffer, 0.1% (v/v) L-proline, 0.1% (v/v) ascorbic acid, 0.4 μg/mL of dexamethasone, 5 ng/mL of TGF-03, and 1% (v/v) antibiotic-antimycotic. The constructs was placed in the media for 4 weeks. Further, the media was changed in every 3 days. The HA-Alg hydrogel was found to be stiff and progressed to opaque constructs over a time duration of week 4, suggesting that chondrogenic differentiation of the hMSCs occurred and the cells laid down extracellular matrix within the hydrogels. To confirm presence of cartilaginous matrix, collagen type II fluorescent staining was detected in constructs as depicted in FIG. 23. Further, secretion of glycosaminoglycans (GAGs) was observed the HA-Alg hydrogel. The GAG is also a chondrogenic marker. This suggest that the HA-Alg exhibits an excellent supporting biomaterial for chondrogenic differentiation of the hMSCs. After chondrogenic differentiation of the hMSCs at 2-weeks and 4-weeks, the constructs were fixed in 4% formalin for 2 hours at room temperature, then transferred into 30% sucrose at 4 degrees Celsius overnight before embedding in cryogel (FCS 22 Clear, Leica). The constructs were sectioned on a transverse plane up to a thickness of 5 μm. The sections were pre-treated with proteinase K for antigen retrieval for 20 minutes, rinsed with the PBS, incubated with blocking serum for 30 minutes at the room temperature, and incubated with 1:200 rabbit zpolyclonal to collagen type II antibody for 1 hour. The sections were washed and incubated with anti-rabbit secondary antibody conjugated with Texas Red for 1 hour at the room temperature in the dark. Negative control samples were hydrogel sections without primary antibody incubation. All samples were mounted with fluoroshield mounting medium with 4′,6-diamidino-2-phenylindole (DAPI). The sections were visualized under an inverted fluorescence microscope.

Experimental data for cytotoxicity of the HA-Al₂ hydrogel and the HA-Al₂ hydrogel encapsulated with the SF

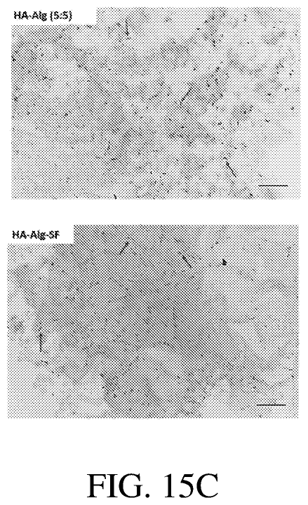

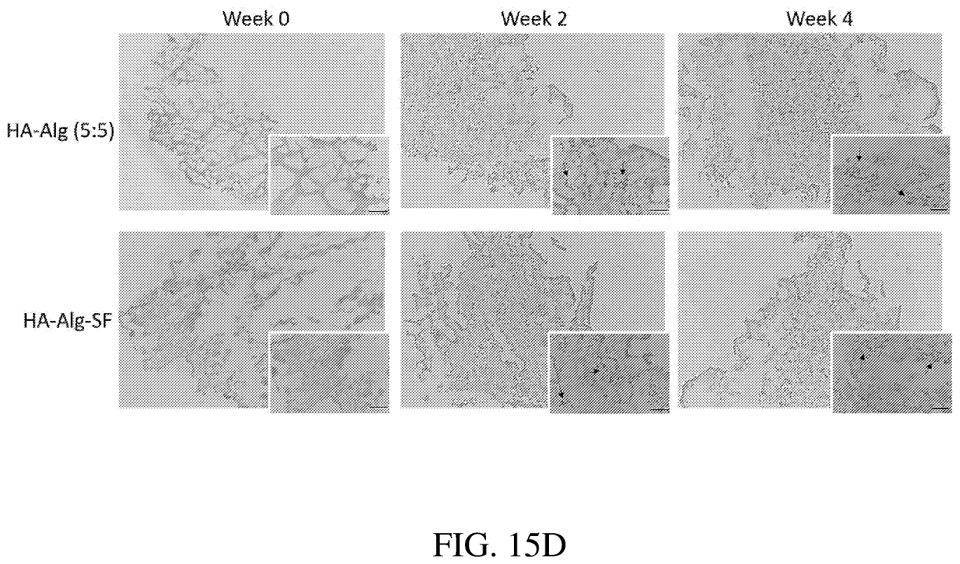

Cytotoxicity and cell distribution of the HA-Alg hydrogel and the HA-Alg hydrogel encapsulated with the SF was demonstrated using LIVE/DEAD cell viability assay. The cytotoxicity was examined after one day, three days and seven days of encapsulation of the hMSC. Three HA-Alg hydrogels and the HA-Alg hydrogels encapsulated with the SF were incubated in Calcein acetoxymethyl (Calcein AM)/ethdium homodimer-1 (EthD-1) for 30 minutes in the dark as per standard procedure. The HA-Alg hydrogels and the HA-Alg hydrogels encapsulated with the SF were visualized using inverted fluorescence microscope to determine living (green) and dead (red) cells after one day, three days and seven days post-encapsulation. The green color represented living cells and the red color represented dead cells. The hMSCs showed homogeneous distribution in the HA-Alg hydrogel and the HA-Alg hydrogel encapsulated with the SF as depicted in FIG. 15A and FIG. 15B respectively. DNA content of both the hydrogels slightly increased up to seven days, including DNA content of the fibrin hydrogel. The fibrin hydrogel consisted of gelatin, fibrinogen, the HA, glycerol, and thrombin. The fibrin hydrogel was used as the control group. The fibrin hydrogel was used as the control group to assess tissue development of the HA-Alg hydrogel and HA-Alg hydrogel encapsulate with the SF. Reported from previous study, the fibrin control group showed excellent biologically acitivites for many cell types and tissues. In addition, immunohistochemistry analysis was performed to determine Ki-67 protein in active phases of the cell cycle, such as G1, S, G2 and mitosis. The immunohistochemistry of the Ki-67 protein is depicted in FIG. 15C. The Ki-67 protein was detected in the HA-Alg hydrogel (having the predefined concentration of 5:5 of the HA-NH₂ solution and the Alg-CHO solution) and the HA-Alg hydrogel encapsulated with the SF as depicted in FIG. 15D. FIG. 15D represents an Alcian blue staining of GAGs secretion in the HA-Alg hydrogel based bioink and the HA-Alg hydrogel encapsulated with the SF at 0 week, and 2 weeks, and 4 weeks (scale bars: 100 μm). Dark blue areas (arrows) represent the secreted GAGs from differentiated chondeocytes detected on week 2 and week 4. The light blue background staining corresponds with glycosaminoglycan of HA, which is the component of HA-Alg hydrogel.

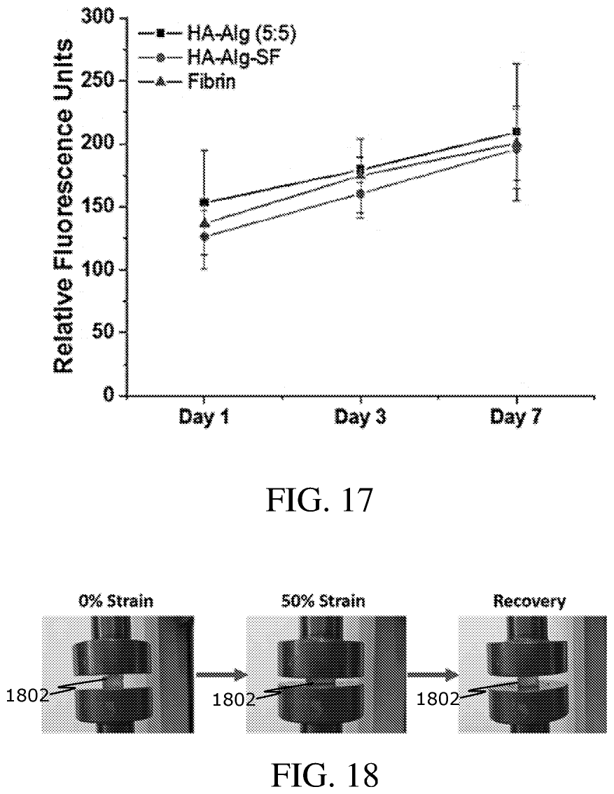

Further, the cytotoxicity of the HA-Alg hydrogel and the HA-Alg hydrogel encapsulated with the SF was investigated using a resorufin-based solution. Resorufin is a red fluorescent compound generated from mitochondrial reductase enzyme of active cells, that reflects cellular metabolism. The HA-Alg hydrogel and the HA-Alg hydrogel encapsulated with the SF were incubated in 1 mL of 1× PrestoBlue™ diluted in medium, as per the standard procedure. The HA-alg hydrogels were incubated at the temperature of 37 degree Celsius, at 5% CO2 for three hours. Following incubation, the medium was taken out and transferred to a ninety-six well plate. Fluorescence of samples was measured at λex 560 nm and λem 590 nm using a microplate reader. The HA-Alg hydrogel encapsulated with the SF was used as a control due to its compatibility for cell encapsulation. Wells included HA-Alg hydrogel (5:5) with the hMSC, HA-Alg hydrogel encapsulated with the SF and the hMSC, and HA-Alg hydrogel without the hMSC. Both hydrogels (the HA-Alg hydrogel and the HA-Alg hydrogel encapsulated with the SF) showed an increase in fluorescence unit throughout the seven days culture period, indicating non-toxicity of the HA-Alg hydrogel against the hMSCs. The fluorescent values corresponded with resorufin, a red fluorescent compound generated from mitochondrial reductase enzyme of active cells, that reflects cellular metabolism is depicted in FIG. 17. These studies demonstrated that HA both hydrogels supported cell proliferation without cytotoxicity.

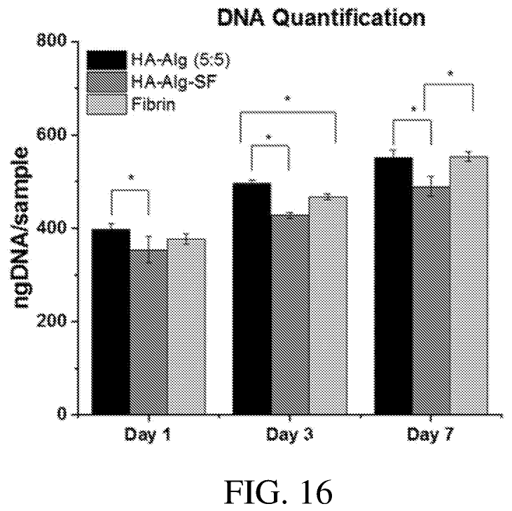

Further, the HA-Alg hydrogel and the HA-Alg hydrogel encapsulated with the SF were removed from culture medium, minced and digested in 1 mL of 1 mg/mL proteinase K containing 20 μL papain solution. DNA content was measured using DNA Assay Kit according to standard protocol. Samples were measured at λex 480 nm and λem 530 nm using a microplate reader. Results confirmed cell proliferation in tissue constructs. FIG. 16 depicts a variation of DNA content of HA-Alg hydrogel, HA-Alg hydrogel encapsulated with the SF and the fibrin hydrogel. Specifically, HA-Alg hydrogel had a comparable DNA content as compared to the fibrin hydrogel but showed a greater value than HA-Alg hydrogel encapsulated with the SF on day 7. Further, it was established that the hMSCs encapsulated in the HA-Alg hydrogel and the HA-Alg hydrogel encapsulated with the SF could transform hydrogel from transparent to opaque constructs by 4 weeks. Cartilaginous matrix production by the hMSCs was confirmed by collagen type II staining in both hydrogels.

### Experimental Data for Mechanical Assessment of the HA-Alg Hydrogel

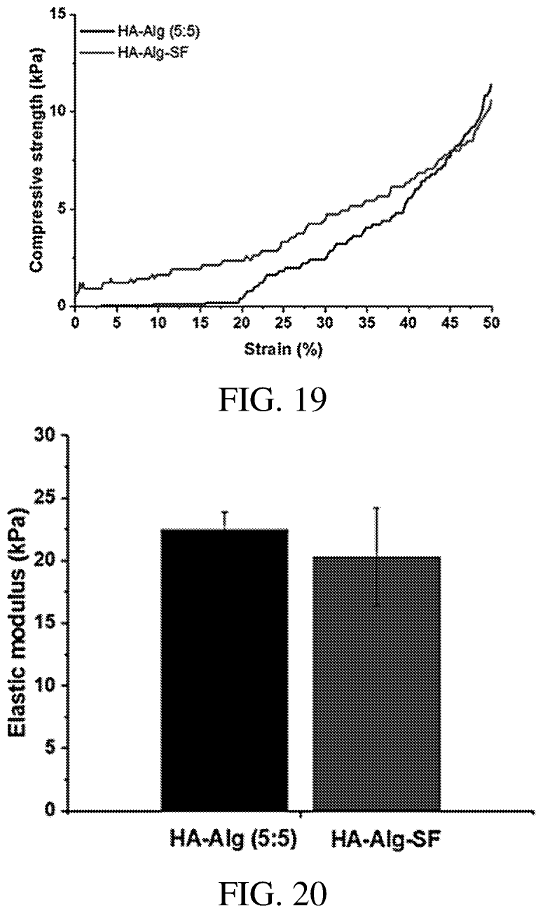

Mechanical properties of the HA-Alg hydrogel and the HA-Alg SF was tested by an electronic testing machine at a room temperature. In this regard, the HA-Alg hydrogel and the HA-Alg SF hydrogel having a diameter of 13 mm and a thickness of 10 mm were placed in the testing machine and subjected to a constant velocity of 10 mm/min in compressive mode. The HA-Alg hydrogel was compressed to 50% of its original thickness. Under compressive loading, the HA-Alg hydrogel and the HA-Alg hydrogel encapsulated with the SF were able to withstand load at 50% strain and recovered to their original shapes as depicted in FIG. 18. The compressive strengths of the HA-Alg hydrogel was found to be 11.44±1.80 kPa and the HA-Alg hydrogel encapsulated with the SF was found to be 10.61±0.36 kPa, at the strain of 50%. Variation of the compressive strength with strain is depicted in FIG. 19. Further, magnitude of the elastic modulus of two hydrogels is depicted in FIG. 20. Incorporation of SF did not show significant difference in elastic modulus between the two hydrogels as shown in table 1 given below.

TABLE 1

| | Compressive modulus | |
|---|---|---|
| | (kPa) | Elastic modulus (kPa) |
| HA-Alg (5:5) | 11.44 ± 1.80 | 22.5 ± 1.36 |
| HA-Alg-SF | 10.61 ± 0.36 | 20.31 ± 3.90 |

### Experimental Data for Degradation of the HA-Alg Hydrogel

Degradation of the HA-Alg hydrogel was demonstrated at different ratio of the HA-NH₂ and the Alg-CHO. Three, HA-Alg hydrogels were submerged in 1×PBS solution (pH 7.4) at 37 degrees Celsius for 60 days. The HA-Alg hydrogels were removed from PBS solution. Wet weight (Wt) of th HA-Alg hydrogels were recorded at different time to calculate percentage of weight differences, Wd (%), as described in equation below.

$$Wd ( \% ) = ( Wt - W 0 ) / W 0 \times 100$$

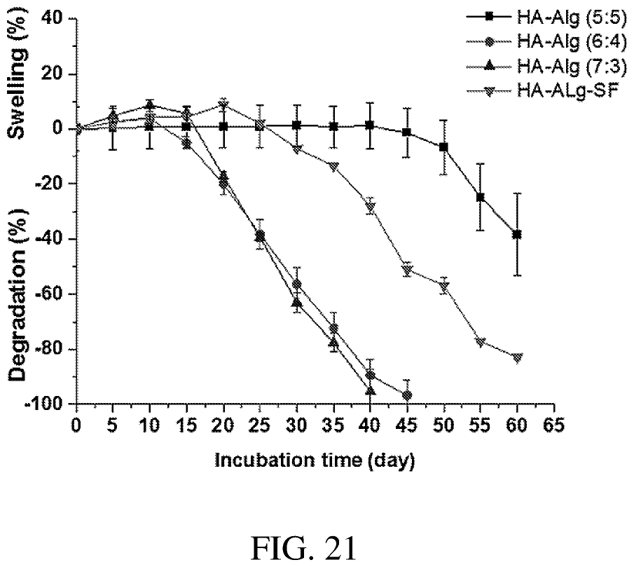

The positive and negative values were expressed in swelling percentage and degradation percentage, respectively. The wet weights of HA-Alg hydrogels were recorded at each different time and compared to the wet weight at day 0. By varying the predefined ratio of the HA-NH₂ solution and the Alg-CHO solution, swelling and degradation of the HA-Alg hydrogels were demonstrated as depicted in FIG. 21. Herein, the HA-Alg hydrogel having the predefined ratio of 7:3 of the HA-NH₂ solution and the Alg-CHO solution, respectively showed a significant increase in the wet weight (swelling) on day 5 and day 10, which were 9.7±1.1% and 13.8±1.9%, respectively. The HA-Alg hydrogel, having the predefined ratio of 7:3 of the HA-NH₂ solution and the Alg-CHO solution, respectively exhibited fastest weight loss and reached 75.7 10±8.4% weight in 35 days. The HA-Alg hydrogel having the predefined ratio of 6:4 of the HA-NH₂ solution and the Alg-CHO solution, respectively showed slight increase in wet weight up to day 10 (1.96±1.5%) and decreased to 80.6±9.8% in 40 days. Swelling and degradation profile of the HA-Alg hydrogel having the predefined ratio of 5:5 of the HA-NH₂ solution and the Alg-CHO solution, respectively remained constant up to 35 days, and slowly degraded to 35.7±13.4% on day 60. Based on the above, the HA-Alg hydrogel at the predefined concentration showed significant stability. The Wd (%) of the HA-Alg hydrogels at different ratio of the NA-HN2 and the Alg-CHO in PBS for 60 days are provided in table 2 given below.

TABLE 2
Weight difference (%)

| Day | HA-Alg (7:3) | HA-Alg (6:4) | HA-Alg (5:5) | HA-Alg-SF |
|---|---|---|---|---|
| 1 | 0.0 | 0.0 | 0.0 | 0.0 |
| 5 | 4.8 ± 2.5 | 2.5 ± 2.5 | 0.3 ± 7.8 | 2.6 ± 1.7 |
| 10 | 8.6 ± 2.1 | 3.8 ± 2.4 | 0.7 ± 6.9 | 4.3 ± 1.6 |
| 15 | 5.6 ± 2.3 | −5.1 ± 2.6 | 0.7 ± 7.4 | 4.4 ± 1.9 |
| 20 | −17.2 ± 1.6 | −19.9 ± 3.7 | 0.8 ± 7.6 | 8.7 ± 2.4 |
| 25 | −39.3 ± 1.4 | −38.3 ± 4.1 | 0.9 ± 7.7 | 2.1 ± 1.04 |
| 30 | −63.1 ± 3.2 | −56.3 ± 4.9 | 1.2 ± 7.3 | −7.1 ± 1.3 |
| 35 | −77.6 ± 3.4 | −72.3 ± 5.8 | 0.8 ± 7.5 | −13.4 ± 1.1 |
| 40 | −95.4 ± 8.0 | −89.4 ± 5.3 | 1.1 ± 8.3 | −27.9 ± 3.0 |
| 45 | | −96.7 ± 5.6 | −1.4 ± 8.8 | −50.9 ± 2.4 |
| 50 | | | −6.8 ± 9.8 | −56.8 ± 3.1 |
| 55 | | | −24.9 ± 11.9 | −77.2 ± 0.6 |
| 60 | | | −38.3 ± 14.9 | −82.8 ± 0.8 |

Experimental Data for Rheological Analysis of the HA-Alg Hydrogel and the HA-Alg Hydrogel Encapsulated with the SF

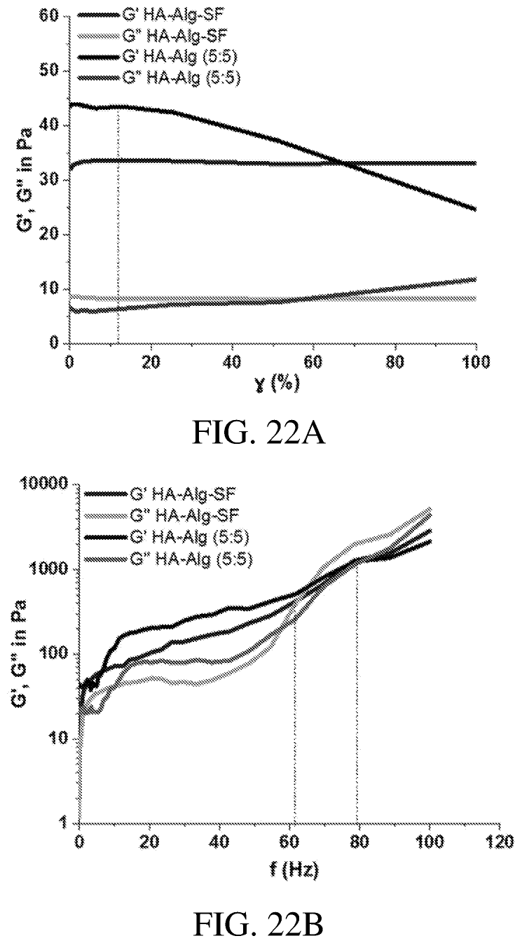

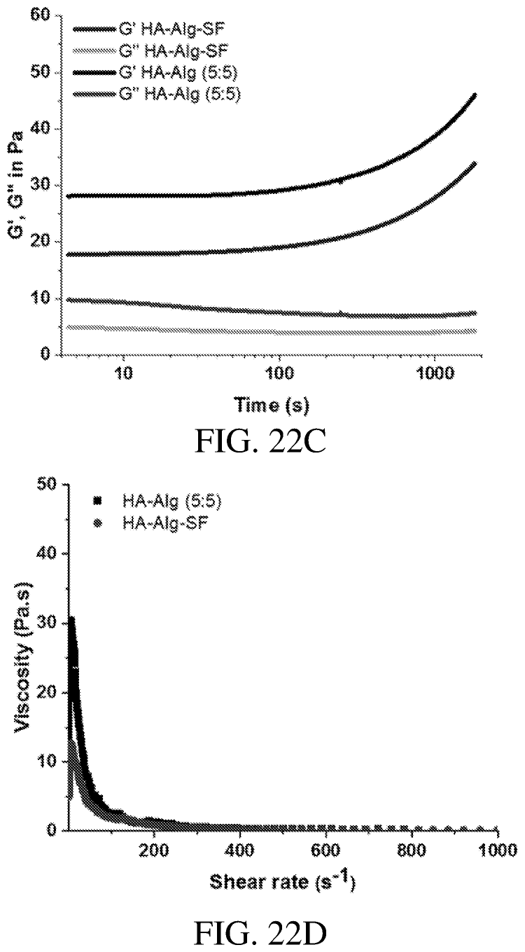

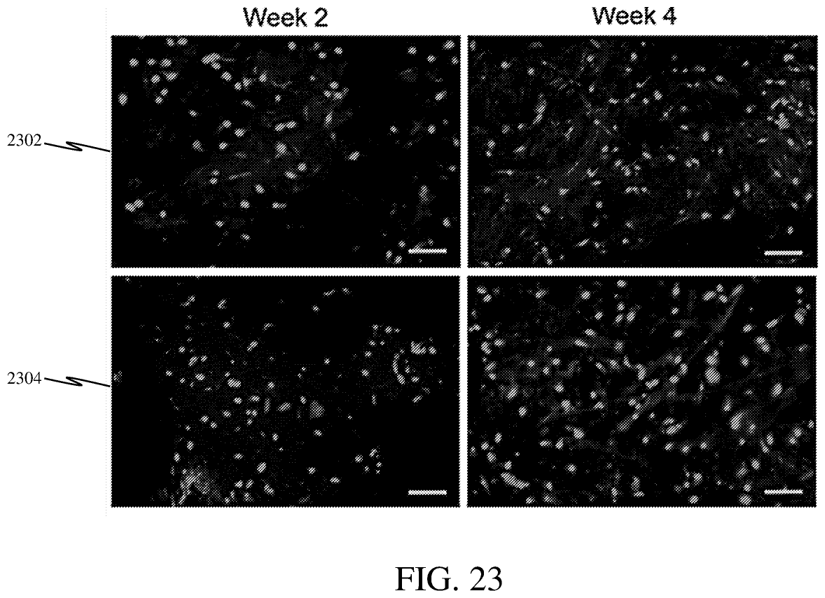

The rheological properties of the HA-Alg hydrogel (5:5) and the HA-Alg hydrogel encapsulated with the SF hydrogels were characterized by a strain sweep experiment, a frequency sweep experiment, a time sweep experiment, and shear-thinning behaviours. Rheological measurements of the HA-Alg hydrogel and the HA-Alg hydrogel encapsulated with the SF were performed with a rheometer. The rheometer was equipped with a set of parallel plates in an oscillatory mode at the room temperature. Strain sweep experiment was conducted over a range of 0.1% to 100%, at a frequency of 1 Hz to determine the linear viscoelasticity region (LVER) of the HA-Alg hydrogel and the HA-Alg hydrogel encapsulated with the SF, which indicated the range of strain amplitudes that do not destroy the hydrogel structure. Herein, the strain sweep experiment from 0.1% to 100% was conducted at a frequency of 1 Hz on the HA-Alg hydrogel and the HA-Alg hydrogel encapsulated with the SF. Then, dependence/independence of elastic modulus (G′) and viscous modulus (G″) over the range of oscillation frequencies (0.01 Hz to 100 Hz) was acquired at the defined strain rate. The storage (G′) and loss (G″) moduli were found beyond 100% for the HA-Alg hydrogel (5:5) and HA-Alg hydrogel encapsulated with the SF as depicted in FIG. 22A. This study suggested that the HA-Alg hydrogel (5:5) and the HA-Alg hydrogel encapsulated with the SF exhibited high stretchability and had large deformation. The frequency sweeps from 0.01 Hz to 100 Hz were carried out at 10% strain, corresponding with the determined LIVE strain amplitude as depicted in FIG. 22B. Crossover points between G′ and G″ for the HA-Alg hydrogel (5:5) and the HA-Alg hydrogel encapsulated with the SF were 78 Hz (black and red lines) and 61 Hz (blue and green lines), respectively. Below these points, the elastic behavior dominated the properties of the hydrogels (G′>G″). On the other hand, exceeding the crossover point, both hydrogels showed a more fluid-like behaviour at high frequency, suggesting that the high frequency interrupted structure of the hydrogels. Both hydrogels, G′ and G″ showed frequency dependence throughout the range test, 0.01 Hz to 100 Hz.

Time sweep experiment was performed to determine the gelation time at 1 Hz, for 30 minutes and at the room temperature. The viscosity of the HA-Alg hydrogel and the HA-Alg hydrogel encapsulated with the SF over a shear rate lying in a range of 0.001 s⁻¹ to 1000 s⁻¹ was recorded.

Time sweep of the HA-Alg hydrogel (5:5) and the HA-Alg hydrogel encapsulated with the SF was expected to provide information of gelation time. In principle, the gelation of hydrogel has been observed by the crossover time between storage (G′) and loss (G″) moduli. Before the gelation, G″ is greater than G′ which shows the liquid behaviour. When the gelation occurs, G′ is equal to G″ and ultimately exceeds G″ value according to time. Although, gelation points of the HA-Alg hydrogel (5:5) and the HA-Alg-SF hydrogel encapsulated with the SF were not detected, G′ was greater than G″ in both hydrogels indicating the elastic behavior of crosslinked hydrogels as depicted in FIG. 22C. The absence of gelation point could be explained by the quick reaction of HA-NH₂ and Alg-CHO before loading into the plate geometry of the rheometer.

For the HA-Alg hydrogel (5:5) and the HA-Alg hydrogel encapsulated with the SF, the viscosity decreased when shear rate increased is depicted in FIG. 22D. Highest viscosity of each hydrogels represents the point where the solid-like elastic state breaks down and transforms to fluid-like state. The HA-Alg hydrogel (5:5) exhibited higher viscosity of 30.4±0.83 Pa·s compared to the viscosity of the HA-Alg hydrogel encapsulated with the SF hydrogel, which was found to be 12.6±1.27 Pa·s.

A decrease in viscosity was observed at the shear rate higher than 9.1 s⁻¹ in the HA-Alg hydrogel (5:5) and higher than 6.6 s⁻¹ in the HA-Alg hydrogel encapsulated with the SF, indicating their remarkable shear-thinning behaviour as depicted in FIG. 22D. The shear-thinning properties of both the hydrogels indicated that they readily passed through the nozzle during printing process, while retained the gel state after extruding. Correspondingly, shear stress (i) applied on the HA-Alg hydrogel (5:5) and the HA-Alg hydrogel encapsulated with the SF was determined by a relationship between apparent viscosity (q) and shear rate (y) as follows:

$$τ = η \dot{γ}$$

The shear stress (τ) at a maximum viscosity of the HA-Alg hydrogel (5:5) and the HA-Alg hydrogel encapsulate with the SF were 276.6 Pa and 83.2 Pa, respectively. When the shear rate reached 1000 s⁻¹, the viscosity of the HA-Alg hydrogel and the HA-Alg hydrogel encapsulate with the SF was maintained at 0.12 Pa·s and 0.13 Pa·s, which corresponded to the shear stress (σ) of 120 Pa and 130 Pa, respectively. A high shear stress (i) can affect both cell viability and cell proliferation. A data presented in rheological analysis and cell viability after printing suggested that the viscosity and shear stress (i) of both hydrogels were within acceptable ranges for printing cells using our custom-made 3D bioprinter. Higher spatial resolution of printed HA-Alg hydrogel over the HA-Alg hydrogel encapsulated with the SF, could be explained by approximately 2.4-times higher viscosity of the HA-Alg hydrogel compared to the HA-Alg hydrogel encapsulated with the SF, suggesting that the SF possibly interfered with HA-Alg network. To improve viscosity of the HA-Alg hydrogel encapsulated with the SF, increasing in degree of crosslinking might reinforce the HA-Alg hydrogel network and improve shear-thinning of the hydrogel incorporated with other polymeric molecules not limited to SF. The rheological characteristics of the HA-Alg hydrogel (5:5) and the HA-Alg hydrogel encapsulate with the SF at the room temperature with 1% strain and at the frequency 1 Hz is mentioned below in table 3.

TABLE 3

| | | | Shear- | | |
|---|---|---|---|---|---|
| | | | thinning | | |
| | | | viscosity | Shear rate | Yield shear |
| | G′ (Pa) | G″ (Pa) | (Pa · s) | (s⁻¹) | stress (Pa) |
| HA-Alg (5:5) | 42.3 ± 1.50 | 9.4 ± 1.31 | 30.4 ± 0.83 | 9.1 | 276.64 ± 5.67 |
| HA-Alg SF | 32.0 ± 1.15 | 4.2 ± 0.07 | 12.6 ± 1.27 | 6.6 | 83.16 ± 6.09 |

### DETAILED DESCRIPTION OF THE DRAWINGS

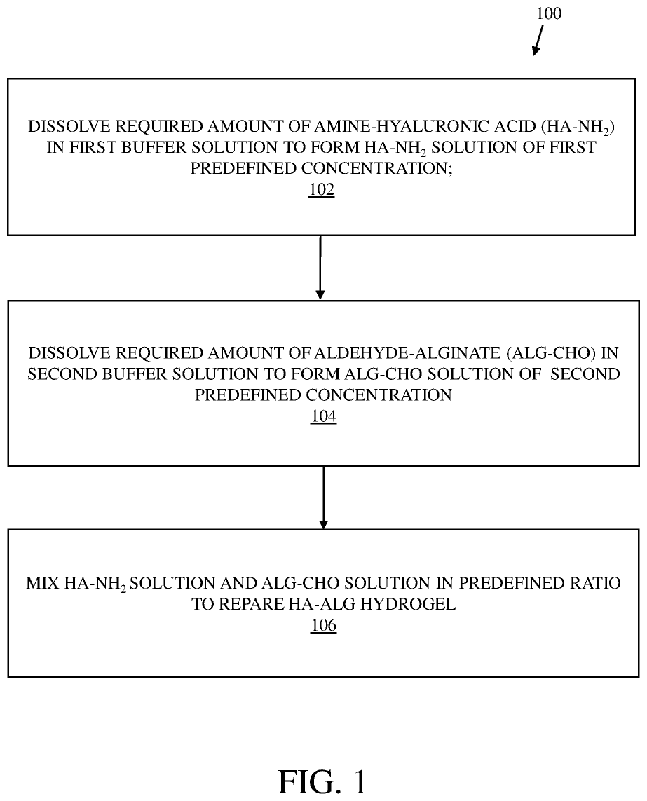

Referring to FIG. 1, illustrated is a flowchart depicting steps of a method for preparing a Hyaluronic acid-Alginate (HA-Alg) hydrogel, in accordance with an embodiment of the present disclosure. At step 102, a required amount of amine-hyaluronic acid (HA-NH₂) is dissolved in a first buffer solution to form an HA-NH₂ solution of a first predefined concentration. At step 104, a required amount of aldehyde-alginate (Alg-CHO) is dissolved in a second buffer solution to form an Alg-CHO solution of a second predefined concentration. At step 106, the HA-NH₂ solution and the Alg-CHO solution is mixed in a predefined ratio for preparing the HA-Alg hydrogel.

The aforementioned steps are only illustrative, and other alternatives can also be provided where one or more steps are added, one or more steps are removed, or one or more steps are provided in a different sequence without departing from the scope of the claims herein.

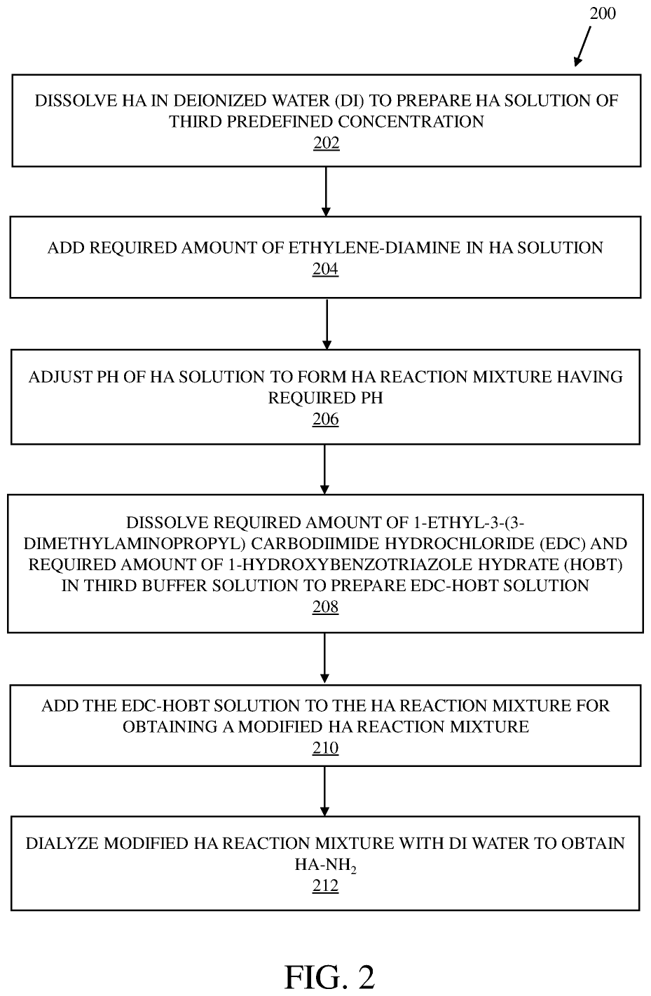

Referring to FIG. 2, illustrated is a flowchart depicting process steps for synthesizing amine-hyaluronic acid (HA-NH₂), in accordance with an embodiment of the present disclosure. At step 202, Hyaluronic acid (HA) is dissolved in deionized water (DI) for preparing a HA solution of a third predefined concentration. At step 204, a required amount of ethylene-diamine is added in the HA solution. At step 206, pH of the HA solution is adjusted to form a HA reaction mixture having a required pH. At step 208, a required amount of 1-ethyl-3-(3-dimethylaminopropyl) carbodiimide hydrochloride (EDC) and a required amount of 1-hydroxybenzotriazole hydrate (HOBt) is dissolved in a third buffer solution for preparing an EDC-HOBt solution. At step 210, the EDC-HOBt solution is added to the HA reaction mixture for obtaining a modified HA reaction mixture. At step 212, the modified HA reaction mixture is dialyzed with the DI water to obtain the HA-NH₂.

The aforementioned steps are only illustrative, and other alternatives can also be provided where one or more steps are added, one or more steps are removed, or one or more steps are provided in a different sequence without departing from the scope of the claims herein.

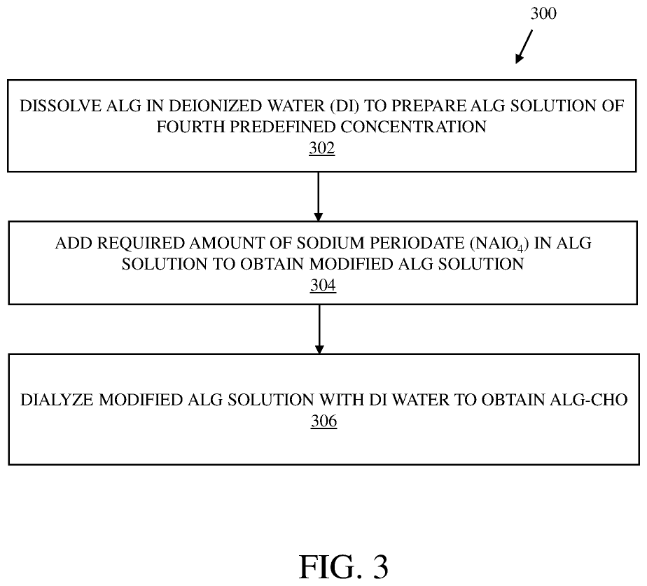

Referring to FIG. 3, illustrated is a flowchart depicting process steps for synthesizing aldehyde-alginate (Alg-CHO), in accordance with an embodiment of the present disclosure. At step 302, Alginate (Alg) is dissolved in deionized (DI) water for preparing an Alg solution of a fourth predefined concentration. At step 304, a required amount of Sodium periodate (NaIO₄) is added to the Alg solution for obtaining a modified Alg solution. At step 306, the modified Alg solution is dialyzed with the DI water for obtaining the Alg-CHO.

The aforementioned steps are only illustrative, and other alternatives can also be provided where one or more steps are added, one or more steps are removed, or one or more steps are provided in a different sequence without departing from the scope of the claims herein.

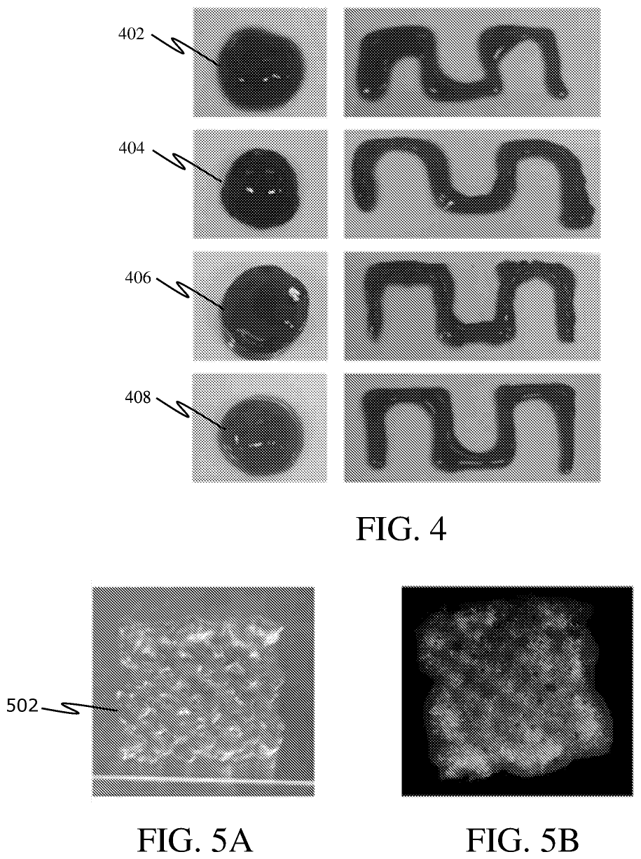

Referring to FIG. 4, illustrated is an exemplary illustration of a Hyaluronic acid-Alginate (HA-Alg) hydrogel (depicted, for example as three HA-Alg hydrogels 402, 404 and 406) at various predefined ratios of Hyaluronic acid-Alginate (HA-NH₂) solution and aldehyde-alginate (Alg-CHO) solution and the HA-Alg hydrogel encapsulated with Silk fibroin (SF), in accordance with an embodiment of the present disclosure. A HA-Alg hydrogel 402 has a predefined ratio of 7:3 of the HA-NH₂ solution and the Alg-CHO solution, respectively. The HA-Alg hydrogel 404 has the predefined ratio of 6:4 of the HA-NH₂ solution and the Alg-CHO solution, respectively. The HA-Alg hydrogel 406 has the predefined ratio of 6:4 of the HA-NH₂ solution and the Alg-CHO solution, respectively. Further, the HA-Alg hydrogel 408 is a hydrogel encapsulated with the SF.

FIG. 4 is merely an example, which should not unduly limit the scope of the claims herein. A person skilled in the art will recognize many variations, alternatives, and modifications of embodiments of the present disclosure.

Referring to FIG. 5A, illustrated is an exemplary illustration of a three-dimensional (3D) construct 502 formed by bioprinting using Hyaluronic acid-Alginate (HA-Alg) hydrogel based bioink, in accordance with an embodiment of the present disclosure. The 3D construct 502 has two layers (not shown) having a grid pattern on each layer. The 3D construct 502 have dimensions of 15 mm×15 mm×0.2 mm.

Referring to FIG. 5B, illustrated is an exemplary illustration of cells viability in the Hyaluronic acid-Alginate (HA-Alg) hydrogel based bioink, in accordance with an embodiment of the present disclosure. Viable cells present in three-dimensional (3D) construct is represented by gray pattern.

Referring to FIG. 6, illustrated is a graphical representation of NMR spectra of amine-hyaluronic acid (HA-NH₂), and aldehyde-alginate (Alg-CHO) in accordance with an embodiment of the present disclosure. Methyl (—CH3) protons of N-acetyl group of hyaluronic acid (HA) and Hyaluronic acid-Alginate (HA-NH₂) showed a signal at 2.06 parts per million (ppm).

Referring to FIG. 7, illustrated are exemplary illustrations of molecular structure of Alginate (Alg), aldehyde-alginate (Alg-CHO) and Hemiacetal, in accordance with an embodiment of the present disclosure. The Hemiacetal is formed as an intermediate product of the Alg-CHO and its hydroxyl groups (—OH).

Referring to FIG. 8, illustrated is a graphical representation of 1H NMR spectra of Alginate (Alg) and aldehyde-alginate (Alg-CHO), in accordance with an embodiment of the present disclosure. The 1H NMR spectra of the Alg and the Alg-CHO exhibited signals at 5.06, 4.66, and 4.48 ppm.

Referring to FIG. 9, illustrated is a graphical representation of 13C NMR spectra of Alginate (Alg) and aldehyde-alginate (Alg-CHO), in accordance with an embodiment of the present disclosure. The 13C NMR spectrum of the Alg-CHO had the new signals at 95.09 ppm and 92.73 ppm.

Referring to FIG. 10A, illustrated is a graphical representation of FTIR spectra of HA, HA-NH₂, Alg and Alg-CHO, in accordance with an embodiment of the present disclosure. Modification of the HA to HA-NH₂ and the Alg to Alg-CHO is represented by peaks at 1552 cm⁻¹ and 1721 cm⁻¹, respectively.

Referring to FIG. 10B, illustrated is a graphical representation of FTIR spectra of Hyaluronic acid (HA), Hyaluronic acid-Alginate (HA-Alg) and Silk fibroin (SF), in accordance with an embodiment of the present disclosure. Gelation of HA-Alg is represented by peak at 1636 cm⁻¹.

Referring to FIG. 11, illustrated is an exemplary illustration of molecular structure of Hyaluronic acid-Alginate (HA-Alg), in accordance with an embodiment of the present disclosure. The HA-Alg is formed by imine bonds between amine group (NH₂) and aldehyde group (CHO).

Referring to FIG. 12, illustrated is an exemplary illustration of a bioprinter (depicted as a bioprinter 1202), in accordance with an embodiment of the present disclosure. The bioprinter 1202 is an extrusion-based bioprinter.

Referring to FIG. 13, illustrated is an exemplary illustration of distribution of fluorescent microspheres, in accordance with an embodiment of the present disclosure. The fluorescent microspheres are distributed homogeneously as represented by gray pattern.

Referring to FIG. 14A, illustrated is an exemplary illustration of a three-dimensional (3D) construct (depicted as a 3D construct 1402) formed by bioprinting using Hyaluronic acid-Alginate (HA-Alg) hydrogel encapsulated with Silk fibroin (SF) and cells, in accordance with an embodiment of the present disclosure. The 3D construct 1402 has two (not shown) having a grid pattern on each layer. The 3D construct 1402 have dimensions of 15 mm×15 mm×0.2 mm.

Referring to FIG. 14B, illustrated is an exemplary representation of cells viability in the Hyaluronic acid-Alginate (HA-Alg) hydrogel, in accordance with an embodiment of the present disclosure. Viable cells present in three-dimensional (3D) construct is represented by gray pattern.

Referring to FIG. 14C, illustrated is an exemplary illustration of a construct (depicted as a construct 1404) formed by Hyaluronic acid-Alginate (HA-Alg) hydrogel, in accordance with an embodiment of the present disclosure. The construct 1404 is shown in an an ear-shaped structure.

Referring to FIGS. 15A-15B, illustrated are exemplary illustrations of distribution of human mesenchymal stem cells (hMSC) in the Hyaluronic acid-Alginate (HA-Alg) hydrogel and the HA-Alg hydrogel encapsulated with Silk-fibroin (SF), respectively, in accordance with an embodiment of the present disclosure. The hMSC are distributed homogeneously as represented by gray dots.

Referring to FIG. 15C, illustrated is an exemplary illustration of immunochemistry of Ki-67 protein in Hyaluronic acid-Alginate (HA-Alg) hydrogel and the HA-Alg hydrogel encapsulated with Silk-fibroin (SF), in accordance with an embodiment of the present disclosure. The immunochemistry of the Ki-67 protein is detected after one week of encapsulation of cells.

Referring to FIG. 15D, illustrated is an exemplary illustration of staining of glycosaminoglycans (GAGs) secretion in the Hyaluronic acid-Alginate (HA-Alg) hydrogel based bioink and the HA-Alg hydrogel encapsulated with Silk fibroin (SF), in accordance with an embodiment of the present disclosure. Secretion of the GAGs represented chondrogenic differentiation of cell in the HA-Alg hydrogel and the HA-Alg hydrogel encapsulated with the SF at zero weeks, two weeks and four weeks.

Referring to FIG. 16, illustrated is graphical representation of DNA content of Hyaluronic acid-Alginate (HA-Alg) hydrogel, HA-Alg hydrogel encapsulated with Silk fibroin (SF) and fibrin hydrogel, in accordance with an embodiment of the present disclosure. The DNA content is detected at one day, third day and seventh day of encapsulation of cells.

Referring to FIG. 17, illustrated is a graphical representation of fluorescence of Hyaluronic acid-Alginate (HA-Alg) hydrogel, HA-Alg hydrogel encapsulated with Silk fibroin (SF) and fibrin hydrogel, in accordance with an embodiment of the present disclosure. The fluorescence is measured at day one, day three and day seven of encapsulation of cells.

Referring to FIG. 18, illustrated is an exemplary illustration of compressive loading of the Hyaluronic acid-Alginate (HA-Alg) hydrogel (depicted as a HA-Alg hydrogel 1802), in accordance with an embodiment of the present disclosure. The HA-Alg hydrogel 1802, when subjected to 50% strain is recovered to its original shape.

Referring to FIG. 19, illustrated is a graphical representation of variation of compressive strength with respect to strain of Hyaluronic acid-Alginate (HA-Alg) hydrogel and HA-Alg hydrogel encapsulated with Silk fibroin (SF), in accordance with an embodiment of the present disclosure. The compressive strength of the HA-Alg hydrogel and the HA-Alg hydrogel encapsulated with the SF is similar at 50% strain.

Referring to FIG. 20, illustrated is a graphical representation of elastic modulus of Hyaluronic acid-Alginate (HA-Alg) hydrogel and HA-Alg hydrogel encapsulated with Silk fibroin (SF), in accordance with an embodiment of the present disclosure. The elastic modulus of the HA-Alg hydrogel and the HA-Alg hydrogel encapsulated with SF is approximately equivalent.

Referring to FIG. 21, illustrated is a graphical representation of variation of swelling profile and degradation profile of Hyaluronic acid-Alginate (HA-Alg) hydrogel at different predefined ratios and HA-Alg hydrogel encapsulated with Silk fibroin (SF), in accordance with an embodiment of the present disclosure. The swelling profile and the degradation profile of the HA-Alg hydrogel at different predefined ratio and the HA-Alg hydrogel encapsulated with SF is represented up to sixty-five days of incubation in Phosphate Buffered Saline (PBS).

Referring to FIG. 22A, illustrated is a graphical representation of variation of elastic modulus (G′) and viscous modulus (G″) of Hyaluronic acid-Alginate (HA-Alg) hydrogel and the HA-Alg hydrogel encapsulated with Silk fibroin (SF) with respect to strain, in accordance with an embodiment of the present disclosure. Rheological properties of the HA-Alg hydrogel at a predefined ratio of 5:5 of amine-hyaluronic acid (HA-NH₂) solution and aldehyde-alginate (Alg-CHO) solution, respectively are characterized by strain sweep experiment.

Referring to FIG. 22B, illustrated is a graphical representation of variation of elastic modulus (G′) and viscous modulus (G″) of Hyaluronic acid-Alginate (HA-Alg) hydrogel and the HA-Alg hydrogel encapsulated with Silk fibroin (SF) with respect to frequency, in accordance with an embodiment of the present disclosure. Rheological properties of the HA-Alg hydrogel at a predefined ratio of 5:5 of amine-hyaluronic acid (HA-NH₂) solution and aldehyde-alginate (Alg-CHO) solution, respectively are characterized by frequency sweep experiment.

Referring to FIG. 22C, illustrated is a graphical representation of variation of elastic modulus (G′) and viscous modulus (G″) of Hyaluronic acid-Alginate (HA-Alg) hydrogel and the HA-Alg hydrogel encapsulated with Silk fibroin (SF) with respect to time, in accordance with an embodiment of the present disclosure. Rheological properties of the HA-Alg hydrogel at a predefined ratio of 5:5 of amine-hyaluronic acid (HA-NH₂) solution and aldehyde-alginate (Alg-CHO) solution, respectively are characterized by time sweep experiment.

Referring to FIG. 22D, illustrated is a graphical representation of variation of viscosity of Hyaluronic acid-Alginate (HA-Alg) hydrogel and the HA-Alg hydrogel encapsulated with Silk fibroin (SF) with respect to shear rate, in accordance with an embodiment of the present disclosure. The viscosity of the HA-Alg hydrogel at a predefined concentration of 5:5 of amine-hyaluronic acid (HA-NH₂) solution and aldehyde-alginate (Alg-CHO) solution and the HA-Alg hydrogel encapsulated with SF decreases when the shear rate increases.

Referring to FIG. 23, illustrated are exemplary illustrations of Immunofluorescent staining of type II collagen in Hyaluronic acid-Alginate (HA-Alg) and the HA-Alg hydrogel encapsulated with Silk fibroin (SF) after two weeks and four weeks, in accordance with an embodiment of the present disclosure. The type II collagen is shown to be detected (depicted for example, as immunofluorescent staining pattern 2302) in constructs (not shown) made of the HA-Alg hydrogel at a predefined ratio of 5:5 of amine-hyaluronic acid (HA-NH₂) solution and aldehyde-alginate (Alg-CHO) solution and the construct made of the HA-Alg hydrogel encapsulated with the SF (depicted for example, as immunofluorescent staining pattern 2304). A higher intensity is shown at four weeks as compared to two weeks in the immunofluorescent staining pattern 2302 and the immunofluorescent staining pattern 2304.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of preparing a Hyaluronic acid-Alginate (HA-Alg) hydrogel, the method comprising:
dissolving a required amount of amine-hyaluronic acid (HA-NH₂) in a first buffer solution to form an HA-NH₂ solution of a first predefined concentration;
dissolving a required amount of aldehy de-alginate (Alg-CHO) in a second buffer solution to form an Alg-CHO solution of a second predefined concentration; and
mixing the HA-NH₂ solution and the Alg-CHO solution in a predefined ratio for preparing the HA-Alg hydrogel.

2. The method according to claim 1, further comprising modifying Hyaluronic acid (HA) for synthesizing the HA-NH₂, the method comprising:
dissolving the HA in deionized water (DI) for preparing a HA solution of a third predefined concentration;
adding a required amount of ethylene-diamine in the HA solution;
adjusting pH of the HA solution to form a HA reaction mixture having a required pH;
dissolving a required amount of 1-ethyl-3-(3-dimethylaminopropyl) carbodiimide hydrochloride (EDC) and a required amount of 1-hydroxybenzotriazole hydrate (HOBt) in a third buffer solution for preparing an EDC-HOBt solution;
adding the EDC-HOBt solution to the HA reaction mixture for obtaining a modified HA reaction mixture; and
dialyzing the modified HA reaction mixture with the DI water for obtaining the HA-NH₂.

3. The method according to claim 2 wherein the step of dialyzing the modified HA reaction mixture is performed using a dialysis bag, the dialysis bag has a plurality of pores having a pore size in a range of from 11,000 Dalton to 15,000 Dalton.

4. The method according to claim 1, further comprising modifying Alginate (Alg) for synthesizing the Alg-CHO, the method comprising:
dissolving the Alg in deionized (DI) water for preparing an Alg solution of a fourth predefined concentration;
adding a required amount of Sodium periodate (NaIO₄) in the Alg solution for obtaining a modified Alg solution; and
dialyzing the modified Alg solution with the DI water for obtaining the Alg-CHO.

5. The method according to claim 4, wherein the step of dialyzing the modified Alg solution is performed using a dialysis bag, the dialysis bag has a plurality of pores having a pore size in a range of from 11,000 Dalton to 15,000 Dalton.

6. The method according to claim 1, wherein the predefined ratio of the HA-NH₂ solution and the Alg-CHO solution is one of: 5:5, 6:4, 7:3 respectively.

7. The method according to claim 1, wherein the first buffer solution and the second buffer solution is implemented as a Phosphate buffered saline (PBS) solution.

8. The method according to claim 1, wherein the first predefined concentration lies in a range of 10-30 mg/ml and the second predefined concentration lies in a range of 10-20 mg/ml.

9. The method according to claim 1, wherein the step of mixing the HA-NH₂ solution and the Alg-CHO solution comprises:
mixing a predefined concentration of cells in a required volume of the Alg-CHO solution to form a cell mixture; and
mixing an equal volume of the HA-NH2 solution with the cell mixture for synthesizing a HA-Alg hydrogel based bioink, wherein the HA-Alg hydrogel based bioink is usable in a bioprinter for bioprinting.

10. The method according to claim 1, wherein the step of mixing the HA-NH₂ solution and the Alg-CHO solution comprises:
adding a required volume of the HA-NH2 solution to a required volume of macromolecule solution for synthesizing a HA-NH2 macromolecule solution; and
adding an equal volume of the Alg-CHO solution to the HA-NH₂ macromolecule solution for encapsulating macromolecules in the HA-Alg hydrogel.

11. The method according to claim 1, wherein the step of mixing the HA-NH₂ solution and the Alg-CHO solution comprises:
adding a required concentration of growth factor and/or protein to a required volume of the Alg-CHO solution to form a growth factor and/or protein solution; and
adding an equal volume of the HA-NH2 solution to the growth factor and/or protein solution for encapsulating growth factors and/or proteins in the HA-Alg hydrogel, wherein the growth factors and/or proteins are released controllably.

12. A Hyaluronic acid-Alginate (HA-Alg) hydrogel comprising a amine-hyaluronic acid (HA-NH₂) solution of a first predefined concentration and an aldehyde-alginate (Alg-CHO) solution of a second predefined concentration, wherein the HA-NH₂ solution and the Alg-CHO solution are mixed in a predefined ratio, and wherein the HA-NH₂ solution comprises HA-NH₂ and a first buffer solution, and the Alg-CHO solution comprises Alg-CHO and a second buffer solution.

13. The HA-Alg hydrogel comprising according to claim 12, wherein the predefined ratio of the HA-NH2 solution and the Alg-CHO solution is one of: 5:5, 6:4, 7:3 respectively.

14. The HA-Alg hydrogel according to claim 12, further comprising a predefined concentration of cells mixed in required volume of the Alg-CHO solution to form a cell mixture, wherein an equal volume of the HA-NH2 solution mixed with the cell mixture enables synthesis of a HA-Alg hydrogel based bioink, and wherein the HA-Alg hydrogel based bioink is usable in a bioprinter for bioprinting.

15. The HA-Alg hydrogel according to claim 12, further comprising a required volume of macromolecule solution mixed in a required volume of the HA-NH2 solution to synthesize a HA-NH₂ macromolecule solution, wherein an equal volume of the Alg-CHO solution mixed with the HA-NH₂ macromolecule solution enables encapsulation of macromolecules in the HA-Alg hydrogel.

16. The HA-Alg hydrogel according to claim 12, further comprising a required concentration of growth factors and/or proteins mixed in a required volume of the Alg-CHO solution to form the growth factor and/or protein solution, wherein an equal volume of the HA-NH2 solution mixed with the growth factor and/or protein solution enables encapsulation of growth factors and/or proteins, wherein the growth factors and/or proteins are released controllably.

17. The HA-Alg hydrogel according to claim 12, wherein the HA-Alg hydrogel has a viscosity lying in a range of 30 Pascal-second-32 Pascal-second.
